SEQUENCE LISTING (31 sequences)

SEQ ID NO: 1 - protein, 8 aa, synthetic construct
GTAGLIGQ

SEQ ID NO: 2 - protein, 5 aa, synthetic construct
YIGSR

SEQ ID NO: 3 - protein, 5 aa, synthetic construct
KKKKK

SEQ ID NO: 4 - protein, 8 aa, synthetic construct
GTAGLKKK

SEQ ID NO: 5 - protein, 8 aa, synthetic construct
GTAGLIKK

SEQ ID NO: 6 - protein, 8 aa, synthetic construct
GTAGLKKK

SEQ ID NO: 7 - protein, 8 aa, synthetic construct
GTAGKKKK

SEQ ID NO: 8 - protein, 8 aa, synthetic construct
GTAKKKKK

SEQ ID NO: 9 - protein, 8 aa, synthetic construct
GTKKKKKK

SEQ ID NO: 10 - protein, 8 aa, synthetic construct
GKKKKKKK

SEQ ID NO: 11 - protein, 8 aa, synthetic construct
KKKKKKKK

SEQ ID NO: 12 - protein, 8 aa, synthetic construct
GTAGLIGK

SEQ ID NO: 13 - protein, 8 aa, synthetic construct
GTAGLIKK

SEQ ID NO: 14 - protein, 8 aa, synthetic construct
GTAGLKKK

SEQ ID NO: 15 - protein, 8 aa, synthetic construct
GTAGKKKK

SEQ ID NO: 16 - protein, 8 aa, synthetic construct
GTAKKKKK

SEQ ID NO: 17 - protein, 8 aa, synthetic construct
GTKKKKKK

SEQ ID NO: 18 - skipped

SEQ ID NO: 19 - protein, 13 aa, synthetic construct
GTAGLKKKYIGSR

SEQ ID NO: 20 - protein, 13 aa, synthetic construct
GTAKKKKKYIGSR

SEQ ID NO: 21 - protein, 13 aa, synthetic construct
GTAGLIGQYIGSR

SEQ ID NO: 22 - protein, 13 aa, synthetic construct
GTAGLIGKYIGSR

SEQ ID NO: 23 - protein, 13 aa, synthetic construct
GTAGLIGQKKKKK

SEQ ID NO: 24 - protein, 9 aa, synthetic construct
GTAGLIGQS

SEQ ID NO: 25 - protein, 5 aa, synthetic construct
CCCCC

SEQ ID NO: 26 - protein, 5 aa, synthetic construct
KKKKK

SEQ ID NO: 27 - protein, 13 aa, synthetic construct
GTAGLIKKYIGSR

SEQ ID NO: 28 - protein, 13 aa, synthetic construct
GTAGKKKKYIGSR

SEQ ID NO: 29 - protein, 13 aa, synthetic construct
GTKKKKKKYIGSR

SEQ ID NO: 30 - protein, 13 aa, synthetic construct
KKKKKKKKYIGSR

SEQ ID NO: 31 - protein, 13 aa, synthetic construct
GKKKKKKKYIGSR

(19) United States
(12) Patent Application Publication
(10) Pub. No.: US 2024/0218017 A1
(43) Pub. Date: Jul. 4, 2024

(54) PEPTIDE AMPHIPHILES WITH MODIFIED DEGRADING SEQUENCES AND METHODS OF USE THEREOF

(71) Applicants: UAB RESEARCH FOUNDATION, Birmingham, AL (US); Endomimetics LLC, BIRMINGHAM, AL (US)

(72) Inventors: HO-WOOK JUN, Birmingham, AL (US); BRIGITTA BROTT, Birmingham, AL (US); XIXI ZHANG, Birmingham, AL (US); JUN CHEN, Birmingham, AL (US); BENJAMIN K. ESTEP, Birmingham, AL (US); JENNIFER SHERWOOD, Birmingham, AL (US); PATRICK HWANG, Birmingham, AL (US)

(21) Appl. No.: 18/482,745

(22) Filed: Oct. 6, 2023

Related U.S. Application Data
(60) Provisional application No. 63/378,936, filed on Oct. 10, 2022.

(51) Int. Cl. C07K 7/06; A61K 38/00

(52) U.S. Cl. CPC C07K 7/06; A61K 38/00

(57) ABSTRACT

Disclosed are peptide amphiphiles comprising a hydrophilic peptide sequence and a hydrophobic tail, wherein the hydrophobic tail comprises a moiety having a substituted C16 alkyl chain, wherein the hydrophilic peptide sequence comprises a degrading sequence (DS), wherein the degrading sequence (DS) comprises the amino acid sequence GTAGLIGQ (SEQ ID NO:1) wherein the DS comprises one or more amino acid substitutions. Also disclosed are compositions, liposomes, gels, and medical devices comprising the peptide amphiphiles and methods of use thereof.

### CROSS-REFERENCE TO RELATED APPLICATIONS

This application claims the benefit of U.S. Provisional Patent Application No. 63/378,936, filed Oct. 10, 2022, which is incorporated by reference herein in its entirety.

### STATEMENT REGARDING FEDERALLY SPONSORED RESEARCH

This invention was made with government support under Grant Number 1R01HL163802-01 awarded by the National Institutes of Health. The government has certain rights in this invention.

### REFERENCE TO SEQUENCE LISTING

The Sequence Listing submitted Mar. 14, 2024 as a text file named “21085.0197U2.xml,” created on Mar. 14, 2024, and having a size of 27,174 bytes is hereby incorporated by reference pursuant to 37 C.F.R. § 1.52(e)(5).

### BACKGROUND

Recently, numerous studies have investigated the co-delivery of different therapeutic drugs, including proteins, hydrophobic compounds, and hydrophilic drugs, to improve cancer therapy, stimulate cardiac repair, and prevent atherosclerotic foam cell formation. In addition, it has been reported that the co-delivery of multiple therapeutics offers an excellent opportunity to target different mechanisms for treating diseases, thereby possibly maximizing the therapeutic efficacy, overcoming drug resistance mechanisms, inducing synergistic effects, or decreasing drug toxicity and side effects.

Nitric oxide (NO) plays a vital role in biology, physiology, and pathophysiology, a key signaling molecule in cardiovascular homeostasis. Abnormal NO delivery is strongly associated with the development of CVD. In addition, NO is an endothelium-derived relaxing factor produced in the vascular endothelial cell (VEC), which can regulate vascular tone, lower lipid levels, and inhibit adhesion molecule expressions, platelet aggregation, and vascular smooth muscle cell (VSMC) proliferation. In addition, the endothelium plays an essential role in regulating AVF development. Endothelial-derived NO is a crucial vasodilator and signaling molecule in vascular remodeling. Moreover, NO demonstrates anti-inflammatory effects in some studies. However, unlike the traditional chemical drugs, the metabolites converted from NO are always harmless. Despite NO's beneficial functions, its use in the clinic is limited due to its high reactivity and short diffusion distance. Several groups have synthesized exogenous NO donors, such as S-nitrosothiols (RSNOs), n-diazeniumdiolates (NONOates), and nitrosamines, which can generate NO under specific conditions. However, these exogenous NO donors are commonly administered by systemic delivery, and they suffer from short half-lives and ineffective delivery. In addition, burst release may occur when the NO is released from these exogenous NO donors, possibly resulting in adverse local toxicity. To solve those problems, drug delivery systems have been developed, including nanomaterials such as liposomes, micelles, peptide amphiphiles, and polymer nanoparticles. Moreover, the combination of NO donors with nanomaterials has emerged as a great strategy for spatial-temporal NO release directly at the target site.

### BRIEF SUMMARY

Disclosed herein are peptide amphiphiles comprising multiple hydrophobic amino acids, including glycine (G), alanine (A), leucine (Leu), and isoleucine (I), and tyrosine (Y). Those hydrophobic portions can lead to a solubility issue of peptide amphiphiles in both aqueous and organic solvents and uniform coating by using it for medical devices. Modifications to the hydrophobic region of the disclosed peptide amphiphiles can result in improved peptide amphiphiles.

Disclosed herein are peptide amphiphiles comprising a hydrophilic peptide sequence and a hydrophobic tail, wherein the hydrophobic tail comprises a moiety having a substituted C16 alkyl chain, wherein the hydrophilic peptide sequence comprises a degrading sequence (DS), wherein the degrading sequence (DS) comprises the amino acid sequence GTAGLIGQ (SEQ ID NO:1) wherein the DS comprises one or more amino acid substitutions.

Also disclosed herein are compositions, liposomes, gels, and medical devices comprising the peptide amphiphiles disclosed herein.

Disclosed herein are methods of using the peptide amphiphiles disclosed herein as well as compositions, liposomes, gels, and medical devices comprising the peptide amphiphiles disclosed herein.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the matrix-assisted laser desorption ionization time-of-flight (MALDI-TOF) imaging mass spectroscopy absorption spectrum of peptide amphiphile 11

(PA-C16-GTAGLKKK-YIGSR; SEQ ID NO: 19).

FIG. 2 shows the matrix-assisted laser desorption ionization time-of-flight (MALDI-TOF) imaging mass spectroscopy absorption spectrum of peptide amphiphile 13

(PA-C16-GTAKKKKK-YIGSR (SEQ ID NO: 20)).

FIG. 3 shows the transmission electron microscopy (TEM) images of peptide amphiphile 11's (PA-C16-GTAGLKKK-YIGSR (SEQ ID NO:19)) self-assembling nanofibers at 21000× and 52000× direct magnification.

FIG. 4 shows the transmission electron microscopy (TEM) images of peptide amphiphile 13's (PA-C16-GTAKKKKK-YIGSR (SEQ ID NO:20)) self-assembling nanofibers at 21000× and 52000× direct magnification.

FIG. 5 shows representative images of solubility of PA-C16-GTAGLIGQ-YIGSR (SEQ ID NO:21), PA-C16-GTAGLKKK-YIGSR (SEQ ID NO:19), and PA-C16-GTAKKKKK-YIGSR (SEQ ID NO:20) in deionized H2O at 1 wt. %.

FIG. 6 shows the percent transmittance/opaqueness of 1 wt. % solutions of PA-C16-GTAGLIGQ-S(SEQ ID NO:24), PA-C16-GTAGLIGQ-KKKKK (SEQ ID NO:23), PA-C16-GTAGLIGQ-YIGSR (SEQ ID NO:21), PA-C16-GTAGLIGK-YIGSR (SEQ ID NO:22), PA-C16-GTAGLKKK-YIGSR (SEQ ID NO: 19), and PA-C16-GTAKKKKK-YIGSR (SEQ ID NO:20) in deionized H2O comparative to H2O as a 100% transmittance control.

FIG. 7 shows the dynamic light scattering (DLS) graphs depicting the average particle sizes (nm) and frequency for PA-C16-GTAGLIGQ-YIGSR (SEQ ID NO:21).

FIG. 8 shows the dynamic light scattering (DLS) graphs depicting the average particle sizes (nm) and frequency for PA-C16-GTAGLKKK-YIGSR (SEQ ID NO:19).

FIG. 9 shows the dynamic light scattering (DLS) graphs depicting the average particle sizes (nm) and frequency for PA-C16-GTAKKKKK-YIGSR (SEQ ID NO:20).

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C: D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”. “an”, and “the” include plural reference unless the context clearly dictates otherwise. Thus, for example, reference to “a peptide amphiphile” includes a plurality of such peptide amphiphiles, reference to “the peptide amphiphile” is a reference to one or more peptide amphiphiles and equivalents thereof known to those skilled in the art, and so forth.

As used herein, the term “peptide amphiphile” refers to a peptide compound possessing both a hydrophilic portion (e.g., a hydrophilic peptide sequence moiety) and a hydrophobic tail (e.g., a hydrocarbon moiety). One property typically associated with a peptide amphiphile can be self-assembly.

As used herein, the term “self-assembly” or “self-assembling” refers to the characteristic of a plurality of molecules of a compound in which a disordered system forms a more organized structure or pattern due to specific, local interactions among the molecules themselves without external force. In one aspect, peptide amphiphiles can be self-assembling. In a further aspect, peptide amphiphiles can self-assemble into nanofibers or nanoparticles.

As used herein, the term “hydrophilic peptide sequence” refers to a peptide residue sequence having hydrophilicity properties relative to a hydrocarbon moiety. A hydrophilic peptide sequence can comprise one or more functional peptide sequences (e.g., degradable peptide sequences, nitric oxide donors, and/or cell adhesive ligands).

As used herein, the term “degradation sequence” refers to a sequence of peptide residues that can be degraded by enzymes or hydrolysis under biological conditions.

As used herein, the term “cell adhesive sequence” refers to a sequence of peptide residues capable of operation as adhesive ligands for cells. In one aspect, due to amphilic characteristic of peptide amphiphiles, the disclosed cell adhesive sequences are exposed at the exterior surface of a nanofiber assembly; thus, such cell adhesive sequence can be available for interaction with one or more cells. One example is an “endothelial cell adhesive sequence.” which refers to a peptide sequence that supports endothelial cell adhesion, spreading, migration, and/or growth.

As used herein, the term “nitric oxide producing donor sequence” refers to a peptide residue (e.g., lysine (K) or cysteine (C)) or sequence of peptide residues (e.g., polylysine (KKKKK; SEQ ID NO:3) or polycysteine (CCCCC; SEQ ID NO:25)) capable of reversibly binding nitric oxide gas, or equivalent thereof) as a complex (e.g., diazoniumdiolates). Thus, the peptide or sequence can serve as a reservoir for nitric oxide gas and can selectively release nitric oxide over time. It is understood that the term can include other nitric oxide doners, for example, any peptide sequences containing cysteine or amine groups.

As used herein, the term “NO-releasing nanomaterials” refers to materials whose size is less than 1 micron and can generate and release nitric oxide to their surrounding environment.

As used herein, the term “degradation sequence” refers to a sequence of peptide residues that can be degraded by enzymes or hydrolysis under biological conditions.

As used herein, the term “subject” refers to a target of administration. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and new born subjects, as well as fetuses, whether male or female, are intended to be covered. A patient refers to a subject afflicted with a disease or disorder. The term “patient” includes human and veterinary subjects.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically: that is, administered to treat an existing disease or condition. In further various aspects, a preparation can be administered prophylactically: that is, administered for prevention of a disease or condition.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder: preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “pharmaceutically active agent” includes a “drug” or an “antibiotic” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention.

As used herein, the terms “delivery” and “delivering” refer to carrying a therapeutic compound or the disclosed compositions to a particular subject site. Delivery can be located in a specific particular location

As used herein, the term “drug delivery systems” refers to materials developed by engineering technologies for delivering therapeutic drugs to specific locations of the subject. These materials are primary particles, such as liposomes, polymeric nanoparticles, micelles, and inorganic nanoparticles.

As used herein, the term “liposomes” refers to a closed vesicle with an internal phase separated by at least one lipid bilayer, whose diameter ranges from 20 nm to 400 nm. One property associated with liposome is that its internal phase and lipid bilayer can hold water-soluble and lipophilic materials. The internal liposome phase means the aqueous region enclosed in the liposome's lipid bilayer. It is also called the “internal water phase” and “liposome internal water phase.” In the present invention, liposomes refer to small single-membrane liposomes (SUV: small unilamellar vesicle).

As used herein, the term “hydrogel” refers to two or multi-component water-swollen biomaterials consisting of a three-dimensional network of polymers and water.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Peptide Amphiphiles

A peptide amphiphile (PA) is a molecule that possesses an amphiphilic structure typically composed of a hydrophilic peptide sequence and a hydrophobic tail. Due to the amphiphilic nature, peptide amphiphiles can self-assemble into various structures, including sheets, spheres, rods, or disks, depending on the charge and environment (pH and salt). In addition. PA is reported to self-assemble into micelles when the PA concentration is above its critical micelle concentration. Moreover, if the hydrophilic head group of the PA is bulkier than the hydrophobic tail, cylindrical micelles known as nanofibers are formed. The driven force for self-assembly is from amino acids of the PA. For instance, the negatively charged amino acids in the backbone of the PA improve the PA's solubility. However, when the negative charge of the amino acids is eliminated by lowing the pH of the PA solution or introducing the divalent ions into the PA system. PA self-assembles into specific stable structures. In addition, it should be noted that the presence of hydrogen bonds among the amino acids of the backbone of the PA leads to the formation of cylindrical structures; otherwise, spherical structures would form.

In some aspects, the hydrophobic tail of the disclosed peptide amphiphiles, comprise a substituted C16 alkyl chain and a degrading sequence. For example, disclosed herein is a peptide amphiphile 2. PA-C16-GTAGLIGQ-YIGSR (SEQ ID NO:21) that comprises multiple hydrophobic amino acids, including glycine (G), alanine (A), leucine (L), and isoleucine (I), and tyrosine (Y). The hydrophobic portions can lead to a solubility issue of peptide amphiphile 2 in both aqueous and organic solvents and ununiform coating by using it for medical devices. Thus, to solve the issue, disclosed herein are peptide amphiphiles with significantly improved solubility. In some aspects, the peptide amphiphiles disclosed herein comprise a sequence wherein one or multiple amino acids of the sequence portion (GTAGLIGQ; SEQ ID NO:1) are substituted with a Lysine (K). In some aspects, the original endothelial adhesive ligand sequence. YIGSR (SEQ ID NO:2) of a peptide amphpiphile can be part of the disclosed peptide amphiphiles. In some aspects, the disclosed peptide amphiphiles can be used with other peptide amphiphiles, such as PA-C16-KKKKK-NO (SEQ ID NO:26), as coating materials to significantly improve the technology based on peptide amphiphile PA-C16-GTAGLIGQ-YIGSR (SEQ ID NO:21) previously known for treating cardiovascular diseases.

Disclosed are peptide amphiphiles, wherein the peptide amphiphile comprises at least one substitution in a degrading sequence (DS) compared to peptide amphiphile 2 (PA-C16-GTAGLIGQ-YIGSR (SEQ ID NO:21)). In some aspects, the peptide amphiphiles can be a modified version of any of the peptide amphiphiles having a DS described in US Patent Application Publication No 2010/0119573, which is incorporated by reference in its entirety herein for its teaching of peptide amphiphiles. Thus, in some aspects, the peptide amphiphiles having at least one substitution in a degrading sequence (DS) can be referred to as modified peptide amphiphiles.

Disclosed are peptide amphiphiles comprising a hydrophilic peptide sequence and a hydrophobic tail, wherein the hydrophobic tail comprises a moiety having a substituted C16 alkyl chain, wherein the hydrophilic peptide sequence comprises a degrading sequence (DS), wherein the degrading sequence (DS) comprises the amino acid sequence GTAGLIGQ (SEQ ID NO: 1) wherein the DS comprises one or more amino acid substitutions. In some aspects, the one or more amino acid substitutions of SEQ ID NO: 1 can be any amino acid substitution. In some aspects, the amino acid substitution of SEQ ID NO: 1 can be a negatively charged amino acid or a polar uncharged amino acid. In some aspects, the amino acid substitution of SEQ ID NO: 1 can be arginine (Arg) and histidine (His), aspartic acid (Asp) and glutamic acid (Glu), serine (Ser), threonine (Thr), asparagine (Asn) and glutamine (Gln), and cysteine (Cys).

Examples of peptide amphiphiles include, but are not limited to peptide amphiphiles comprising PA-C16-DS, PA-C16-CA, PA-C16-K5, PA-C16-K5-NO. PA-C16-YK, and PA-C16-YK5-NO. Each of which is described below.

Peptide amphiphile 1 (PA-C16-DS) comprises a hydrophilic peptide sequence and a hydrophobic tail, wherein the hydrophobic tail comprises a moiety having optionally substituted C16 alkyl chain, wherein the hydrophilic peptide sequence comprises a degrading sequence (DS) comprising an amino acid sequence Gly-Thr-Ala-Gly-Leu-ILE-Gly-Gln (GTAGLIGQ; SEQ ID NO:1), wherein the degradation sequence comprises a matrix metalloprotease (MMP) specific cleavage site.

Peptide amphiphile 2 (PA-C16-CA) comprises a hydrophilic peptide sequence and a hydrophobic tail, wherein the hydrophobic tail comprises a moiety having optionally substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and also comprises a degrading sequence (DS) comprising an amino acid sequence Gly-Thr-Ala-Gly-Leu-ILE-Gly-Gln (GTAGLIGQ; SEQ ID NO:1).

Peptide amphiphile 3 (PA-C16-K5) comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1), wherein herein one or more of the lysine residues comprise pendant amine groups that react with NO to form a diazeniumdiolate-modified peptide amphiphile, named as peptide amphiphile 4 (PA-C16-K5-NO).

Peptide amphiphile 5 (PA-C16-YK) comprises peptide amphiphile 2 (PA-C16-CA) and peptide amphiphile 3 (PA-C16-K5) are at a molar ratio of from about 1:20 to about 20:1, including about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20; wherein herein one or more of the lysine residues comprising pendant amine groups of peptide amphiphile 5 can react with NO to form a diazeniumdiolate-modified peptide amphiphile, named as peptide amphiphile 6 (PA-C16-YK5-NO), also known an endothelium mimicking nanomatrix.

Disclosed herein are peptide amphiphiles comprising PA-C16-DS, PA-C16-CA, PA-C16-K5. PA-C16-K5-NO. PA-C16-YK, and PA-C16-YK5-NO, wherein the DS comprises one or more amino acid substitutions. In some aspects, the DS comprises the sequence, GTAGLIGK (SEQ ID NO: 12), GTAGLIKK (SEQ ID NO:13), GTAGLKKK (SEQ ID NO:14), GTAGKKKK (SEQ ID NO:15), GTAKKKKK (SEQ ID NO:16), GTKKKKKK (SEQ ID NO:17). GKKKKKKK (SEQ ID NO:10) or KKKKKKKK (SEQ ID NO:11).

In some aspects, the one or more substituted amino acids can be on the N-terminal end. C-terminal end, or in the middle of the DS.

In some aspects, the disclosed peptide amphiphiles having a DS with one or more substitutions comprise a hydrophilic peptide sequence and a hydrophobic tail, wherein the hydrophobic tail comprises a moiety having a substituted C16 alkyl chain, wherein the hydrophilic peptide sequence comprises a degrading sequence (DS) wherein the degrading sequence (DS) comprises an amino acid sequence comprising the sequence X₁X₂X₃X₄X₅X₆X₇X₈; SEQ ID NO:18, wherein any of X1-X8 can be any amino acid. In some aspects, the DS comprises the sequence, GTAGLIGK (SEQ ID NO: 12), GTAGLIKK (SEQ ID NO: 13), GTAGLKKK (SEQ ID NO:14), GTAGKKKK (SEQ ID NO:15), GTAKKKKK (SEQ ID NO:16), GTKKKKKK (SEQ ID NO: 17). GKKKKKKK (SEQ ID NO:10) or KKKKKKKK (SEQ ID NO:11).

In some aspects, the disclosed peptide amphiphiles having a DS with one or more substitutions comprise a hydrophilic peptide sequence and a hydrophobic tail, wherein the hydrophobic tail comprises a moiety having a substituted C16 alkyl chain, wherein the hydrophilic peptide sequence comprises a degrading sequence (DS) wherein the degrading sequence (DS) comprises an amino acid sequence comprising the sequence X₁X₂X₃X₄X₅X₆X₇X₈; SEQ ID NO:18, wherein X1 is Gly, a positive amino acid, a negatively charged amino acid or a polar uncharged amino acid; wherein X2 is Thr, a positive amino acid, a negatively charged amino acid or a polar uncharged amino acid; wherein X3 is Ala, a positive amino acid, a negatively charged amino acid or a polar uncharged amino acid; wherein X4 is r Gly, a positive amino acid, a negatively charged amino acid or a polar uncharged amino acid; wherein X5 is Leu, a positive amino acid, a negatively charged amino acid or a polar uncharged amino acid; wherein X6 is Ile, a positive amino acid, a negatively charged amino acid or a polar uncharged amino acid; wherein X7 is Gly, a positive amino acid, a negatively charged amino acid or a polar uncharged amino acid; and wherein X8 is Gln, a positive amino acid, a negatively charged amino acid or a polar uncharged amino acid. In some aspects, the DS comprises the sequence, GTAGLIGK (SEQ ID NO:12), GTAGLIKK (SEQ ID NO:13), GTAGLKKK (SEQ ID NO:14), GTAGKKKK (SEQ ID NO:15), GTAKKKKK (SEQ ID NO:16), GTKKKKKK (SEQ ID NO:17). GKKKKKKK (SEQ ID NO: 10) or KKKKKKKK (SEQ ID NO:11).

In some aspects, disclosed herein are peptide amphiphiles comprising a hydrophilic peptide sequence and a hydrophobic tail, wherein the hydrophobic tail comprises a moiety having a substituted C16 alkyl chain, wherein the hydrophilic peptide sequence comprises a degrading sequence (DS) wherein the degrading sequence (DS) comprises an amino acid sequence comprising the sequence (X₁X₂X₃X₄X₅X₆X₇X₈; SEQ ID NO:18), wherein X1 is Lys or Gly. X2 is Lys or Thr. X3 is Lys or Ala. X4 is Lys or Gly. X5 is Lys or Leu. X6 is Lys or Ille. X7 is Lys or Gly, and X8 is Lys or Gln. In some aspects, the DS comprises the sequence, GTAGLIGK (SEQ ID NO:12), GTAGLIKK (SEQ ID NO:13), GTAGLKKK (SEQ ID NO:14), GTAGKKKK (SEQ ID NO:15), GTAKKKKK (SEQ ID NO:16), GTKKKKKK (SEQ ID NO:17). GKKKKKKK (SEQ ID NO:10) or KKKKKKKK (SEQ ID NO: 11).

In some aspects, all but one, all but two, all but three, all but four, all but five, all but six, or all but seven of the amino acids of the DS (e.g. SEQ ID NO: 1 or X₁X₂X₃X₄X₅X₆X₇X₈; SEQ ID NO:18) are substituted.

In some aspects, the DS of the disclosed peptide amphiphiles comprises a MMP specific cleavage site.

In some aspects, the disclosed peptide amphiphiles further comprise a cell-adhesive sequence. In some aspects, the cell-adhesive sequence is an endothelial cell adhesive sequence that does not bind to smooth muscle cells and/or platelets. In some aspects, the cell-adhesive sequence comprises the amino acid sequence YIGSR (SEQ ID NO:2).

In some aspects, the peptide amphiphiles comprise a DS, wherein the DS comprises the amino acid sequence GTAGLIGK (SEQ ID NO:12). For example, disclosed is peptide amphiphile PA-C16-GTAGLIGK-YIGSR (SEQ ID NO:22) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Ala-Gly-Leu-Ile-Gly-Lys (SEQ ID NO:4).

In some aspects, the peptide amphiphiles comprise a DS, wherein the DS comprises the amino acid sequence GTAGLIKK (SEQ ID NO:13). For example, disclosed is peptide amphiphile PA-C16-GTAGLIKK-YIGSR (SEQ ID NO:27) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Ala-Gly-Leu-Ile-Lys-Lys (SEQ ID NO:5).

In some aspects, the peptide amphiphiles comprise a DS, wherein the DS comprises the amino acid sequence GTAGLKKK (SEQ ID NO: 14). For example, disclosed is peptide amphiphile PA-C16-GTAGLKKK-YIGSR (SEQ ID NO:19) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Ala-Gly-Leu-Lys-Lys-Lys (SEQ ID NO:6).

In some aspects, the peptide amphiphiles comprise a DS, wherein the DS comprises the amino acid sequence GTAGKKKK (SEQ ID NO:15). For example, disclosed is peptide amphiphile PA-C16-GTAGKKKK-YIGSR (SEQ ID NO:28) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Ala-Gly-Lys-Lys-Lys-Lys (SEQ ID NO:7).

In some aspects, the peptide amphiphiles comprise a DS, wherein the DS comprises the amino acid sequence GTAKKKKK (SEQ ID NO:16). For example, disclosed is peptide amphiphile PA-C16-GTAKKKKK-YIGSR (SEQ ID NO:20) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Ala-Lys-Lys-Lys-Lys-Lys (SEQ ID NO:8) for improving PA solubility.

In some aspects, the peptide amphiphiles comprise a DS, wherein the DS comprises the amino acid sequence GTKKKKKK (SEQ ID NO:17). For example, disclosed is peptide amphiphile PA-C16-GTKKKKKK-YIGSR (SEQ ID NO:29) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Lys-Lys-Lys-Lys-Lys-Lys (SEQ ID NO:9) for improving PA solubility.

In some aspects, the peptide amphiphiles comprise a DS, wherein the DS comprises the amino acid sequence GKKKKKKK (SEQ ID NO:10). For example, disclosed is peptide amphiphile PA-C16-GKKKKKKK-YIGSR (SEQ ID NO:31) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Lys-Lys-Lys-Lys-Lys-Lys-Lys (SEQ ID NO:10) for improving PA solubility.

In some aspects, the peptide amphiphiles comprise a DS, wherein the DS comprises the amino acid sequence KKKKKKKK (SEQ ID NO:11). For example, disclosed is peptide amphiphile PA-C16-KKKKKKKK-YIGSR (SEQ ID NO:30) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and a sequence for improving PA solubility Lys-Lys-Lys-Lys-Lys-Lys-Lys-Lys (SEQ ID NO:11).

Disclosed herein are peptide amphiphiles comprising a hydrophilic peptide sequence and a hydrophobic tail, wherein the hydrophobic tail comprises a moiety having a substituted C16 alkyl chain, wherein the hydrophilic peptide sequence comprises a degrading sequence (DS) wherein the degrading sequence (DS) comprises an amino acid sequence comprising the sequence (X₁X₂X₃X₄X₅X₆X₇X₈; SEQ ID NO: 18), wherein one or more the X1, X2. X3, X4, X5, X6, X7, or X8 are a lysine residue. In some aspects, the DS comprises the sequence, GTAGLIGK (SEQ ID NO: 12), GTAGLIKK (SEQ ID NO:13), GTAGLKKK (SEQ ID NO:14), GTAGKKKK (SEQ ID NO:15), GTAKKKKK (SEQ ID NO:16), GTKKKKKK (SEQ ID NO:17), GKKKKKKK (SEQ ID NO:10) or KKKKKKKK (SEQ ID NO:11). In some aspects, one or more of the Lys residues comprise a pendant amine group. In some aspects, the pendant amine group can react with Nitric Oxide (NO) to form a diazeniumdiolate-modified peptide amphiphile. Thus, in some aspects, the disclosed peptide amphiphiles can further comprise one or more molecules of NO to form NO releasing peptide amphiphiles.

Disclosed is peptide amphiphile PA-C16-GTAGLIGK-YIGSR (SEQ ID NO:22), wherein one or more of the Lys residues of the peptide amphiphile comprise a pendant amine group, wherein the pendant amine group can react with NO to form a diazeniumdiolate-modified peptide amphiphile (PA-C16-GTAGLIGK-YIGSR-NO (SEQ ID NO:22)).

Disclosed is peptide amphiphile PA-C16-GTAGLIKK-YIGSR (SEQ ID NO:27), wherein one or more of the Lys residues of the peptide amphiphile comprise a pendant amine group, wherein the pendant amine group can react with NO to form a diazeniumdiolate-modified peptide amphiphile (peptide amphiphile 10-NO: PA-C16-GTAGLIKK-YIGSR-NO (SEQ ID NO:27))

Disclosed is peptide amphiphile PA-C16-GTAGLKKK-YIGSR (SEQ ID NO:19), wherein one or more of the Lys residues of the peptide amphiphile comprise a pendant amine group, wherein the pendant amine group can react with NO to form a diazeniumdiolate-modified peptide amphiphile (peptide amphiphile 11-NO: PA-C16-GTAGLKKK-YIGSR-NO (SEQ ID NO:19))

Disclosed is peptide amphiphile PA-C16-GTAGKKKK-YIGSR, wherein one or more of the Lys residues of the peptide amphiphile comprise a pendant amine group, wherein the pendant amine group can react with NO to form a diazeniumdiolate-modified peptide amphiphile (peptide amphiphile 12-NO: PA-C16-GTAGKKKK-YIGSR-NO (SEQ ID NO:28))

Disclosed is peptide amphiphile PA-C16-GTAKKKKK-YIGSR (SEQ ID NO:20), wherein one or more of the Lys residues of the peptide amphiphile comprise a pendant amine group, wherein the pendant amine group can react with NO to form a diazeniumdiolate-modified peptide amphiphile (peptide amphiphile 13-NO: PA-C16-GTAKKKKK-YIGSR-NO (SEQ ID NO:20)).

Disclosed is peptide amphiphile PA-C16-GTKKKKKK-YIGSR (SEQ ID NO:29), wherein one or more of the Lys residues of the peptide amphiphile comprise a pendant amine group, wherein the pendant amine group can react with NO to form a diazeniumdiolate-modified peptide amphiphile (peptide amphiphile 14-NO: PA-C16-GTKKKKKK-YIGSR-NO (SEQ ID NO:29))

Disclosed is peptide amphiphile PA-C16-GKKKKKKK-YIGSR (SEQ ID NO:31), wherein one or more of the Lys residues of the peptide amphiphile comprise a pendant amine group, wherein the pendant amine group can react with NO to form a diazeniumdiolate-modified peptide amphiphile (peptide amphiphile 15-NO: PA-C16-GKKKKKKK-YIGSR-NO (SEQ ID NO:31))

Disclosed is peptide amphiphile PA-C16-KKKKKKKK-YIGSR (SEQ ID NO:30), wherein one or more of the Lys residues of the peptide amphiphile comprise a pendant amine group, wherein the pendant amine group can react with NO to form a diazeniumdiolate-modified peptide amphiphile (peptide amphiphile 16-NO: PA-C16-KKKKKKKK-YIGSR-NO (SEQ ID NO:30))

### 1. Nitric Oxide

Nitric oxide (NO) plays an essential role in biology, physiology, and pathophysiology, a key signaling molecule in vessel wall homeostasis. In addition. NO is an endothelium-derived relaxing factor produced in the vascular endothelial cells (VECs), which plays a crucial role in maintaining endothelial function and shows vasoprotective effects for the endothelium. NO production in VECs was achieved by oxidizing the L-arginine and oxygen, catalyzed by nitric oxide synthase. It is also reported that the abnormal alteration of endothelial function is strongly associated with the imbalance between endogenous vasodilator. NO, and other factors such as endothelium-dependent hyperpolarization, the enhanced oxidative stress state, and the generated vasoconstrictors. Moreover. NO also regulates vascular tone, lowers lipid levels, and inhibits adhesion molecule expressions, platelet aggregation, and vascular smooth muscle cell proliferation. In particular. NO released from the endothelium leads to increased concentrations of cyclic guanosine monophosphate (GMP) and cyclic GMP levels in SMCs, thereby resulting in the local relaxation of SMCs is essential for maintaining laminar blood flow for the blood vessel. In addition to SMCs, cyclic GMP in platelet was also increased by NO and suppressed the activation and adhesion of platelets to the endothelium. NO is also known for promoting endothelial cell growth, survival, and migration. Thus, insufficient NO bioavailability in the cardiovascular system may lead to endothelial injury and atherosclerosis development. Meanwhile. NO is a potent mediator of inflammation and immune response, shown to have anti-inflammatory effects. Despite NO's excellent properties, it is challenging to handle NO due to the necessity of complete oxygen exclusion. In addition, the use of NO in the clinic is still limited due to its high reactivity and short diffusion distance. Therefore, the delivery of NO by carriers has been investigated.

Several groups have synthesized exogenous NO donors, such as S-nitrosothiols (RSNOs), n-diazeniumdiolates (NONOates), and nitrosamines, which can generate NO under specific conditions. However, these exogenous NO donors are commonly administrated by systematic delivery, which suffers from short half-lives and ineffective delivery. In addition, burst release may occur when the NO is released from these exogenous NO donors, possibly resulting in adverse local toxicity. Therefore, drug delivery systems have been developed to solve those problems, including nanomaterials such as liposomes, micelles, dendrimers, peptide amphiphiles, inorganic nanoparticles, carbon nanotubes, and polymer nanoparticles. For instance, nontoxic polyamide dendrimers bearing 18 NO-releasing groups showed potent anti-inflammatory activity, as demonstrated by a significant inhibition of IL-8 production. In addition, PAMAM dendrimers were reported to inhibit thrombin-mediated platelet aggregation.

The disclosed NO-associated compound herein from peptide amphiphile 4, and 2 is NONOates. The chemical structure of this compound can be inferred from the name: diazen N═N, ium: formal positive charge, diolate: two negative oxygens.

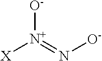

Diazeniumdiolates can be formed by the reaction of a nucleophilic amine (X—) with NO as shown in the following reaction:

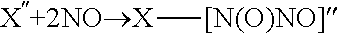

Diazoniumdiolates then dissociate on protonation to release free NO as shown in the following reaction:

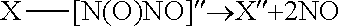

Notably, as shown below, the peptides amphiphile 4, amphiphile 6, and 8 for making the disclosed composition for improving the treatment of cardiovascular diseases such as atherosclerosis have nucleophilic amine on their side chain of the amino acid (lysine). The pendant amine groups of the lysine of the peptide react with NO to form a diazeniumdiolate-modified peptide [K(NO)NO-]]n, wherein “n” is from 1 to 20. As the nitric oxide producing donor sequence of both peptide amphiphile 4 and amphiphile 2 comprise the amino acid sequence Lys-Lys-Lys-Lys-Lys (SEQ ID NO:3), one or more lysine residues comprising the pendant amine group can react with NO to form a diazeniumdiolate-modified peptide [K(NO)NO-]]n, where n can be 1, 2, 3, 4, or 5.

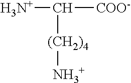

Notably, some earlier studies showed that NO released from polymers possessing lysine could suppress platelet attachment and SMC proliferation, contributing significantly to restenosis.

### 2. Hydrophilic Peptide Sequences and Hydrophobic Tail

Disclosed herein are peptide amphiphiles comprising one or more hydrophilic peptide sequences and a hydrophobic tail. In some aspects, three types of hydrophilic peptide sequences can be present in the disclosed peptide amphiphiles.

In an aspect, the first peptide sequence of the peptide amphiphiles can be a degradation sequence, comprising an amino acid sequence that undergoes cell-mediated proteolytic degradation. The degradation sequence can comprises an MMP2 specific cleavage site, wherein comprises an amino acid sequence Gly-Thr-Ala-Gly-Leu-Ile-Gly-Gln (GTAGLIGQ; SEQ ID NO:1). MMPs are zinc-dependent endopeptidases belonging to a larger family of proteases known as the metzincin Superfamily. The MMPs can be divided into four types, the collagenases, the gelatinases, the stromelysins, and the membrane-type MMPs (MT-MMPs), dependent on the MMPs' substrate specificity and intercellular location. In particular, the collagenases are responsible for degrading triple-helicalcollagens, the significant components of bone and cartilage, into distinctive fragments. The traditional collagenase family includes MMP1. MMP8, MMP13, and MMP18, while MMP2 and MM9 belong to gelatinases. The primary substrates of the gelatinases are type IV collagen and gelatin. In contrast to collagenase, the stromelysins can cleave extracellular matrix proteins but not the triple-helical fibrillar collagens, including MMP3, MMP10, and MMP11. In addition to the discussed MMPs, other MMPs, such as MMP14. MMP15, MMP16. MMP17. MMP24, and MMP25, are MT-MMPs.

In an aspect, endothelial cell adhesive sequences comprise the amino acid sequence of Tyr-Ile-Gly-Ser-Arg (YIGSR; SEQ ID NO:2) can be used. YIGSR (SEQ ID NO:2) is a synthetic laminin-derived pentapeptide. Laminins are a non-collagenous glycoprotein from basement membranes, essential for building a cellular network that connects the intracellular and extracellular components. YIGSR (SEQ ID NO:2) has been shown to improve cell adhesion, regulate myoblast cell function, and promote laminin receptor binding. Incorporating the YIGSR sequence in polyurethane has been shown to enhance endothelial cell adhesion and spreading but inhibit smooth muscle cell proliferation. In some aspects, peptide amphiphiles with YIGSR (SEQ ID NO:2) sequence can significantly improved endothelial cell adhesion, spreading, and proliferation while remarkably reducing platelet adhesion.

In an aspect, a nitric oxide-producing donor sequence comprising an amino acid sequence Lys-Lys-Lys-Lys-Lys (SEQ ID NO:3: KKKKK) can be used.

In some aspects, the hydrophobic tail of the peptide amphiphile can comprise a moiety having an optionally substituted C4 or larger alkyl chain. Thus, the hydrophobic tail can comprise a moiety having an optionally substituted C6 to C28 or a larger alkyl chain. Thus, the hydrophobic tail can comprise a moiety having an optionally Substituted C10 to C25 or larger alkyl chain. Thus, the hydrophobic tail can comprise a moiety having an optionally substituted C4, C5, C6, C7, C8, C9, C10, C11, C12, C13, C14, C15, C16, C17, C18, C19, C20, C21, C22, C23, C24, C25, C26, C27, C28, or larger alkyl chain. Thus, the hydrophobic tail can comprise a moiety having an optionally substituted C16 alkyl chain.

### C. Compositions

Disclosed are compositions comprising one or more of the peptide amphiphiles described herein.

Disclosed are compositions comprising peptide amphiphiles comprising a hydrophilic peptide sequence and a hydrophobic tail, wherein the hydrophobic tail comprises a moiety having a substituted C16 alkyl chain, wherein the hydrophilic peptide sequence comprises a degrading sequence (DS), wherein the degrading sequence (DS) comprises the amino acid sequence GTAGLIGQ (SEQ ID NO:1) wherein the DS comprises one or more amino acid substitutions.

Disclosed are compositions comprising peptide amphiphiles comprising a hydrophilic peptide sequence and a hydrophobic tail, wherein the hydrophobic tail comprises a moiety having a substituted C16 alkyl chain, wherein the hydrophilic peptide sequence comprises a degrading sequence (DS) wherein the degrading sequence (DS) comprises an amino acid sequence comprising the sequence (X₁X₂X₃X₄X₅X₆X₇X₈; SEQ ID NO: 18), wherein X1 is Gly, a positive amino acid, a negatively charged amino acid or a polar uncharged amino acid; wherein X2 is Thr, a positive amino acid, a negatively charged amino acid or a polar uncharged amino acid; wherein X3 is Ala, a positive amino acid, a negatively charged amino acid or a polar uncharged amino acid; wherein X4 is r Gly, a positive amino acid, a negatively charged amino acid or a polar uncharged amino acid; wherein X5 is Leu, a positive amino acid, a negatively charged amino acid or a polar uncharged amino acid; wherein X6 is Ile, a positive amino acid, a negatively charged amino acid or a polar uncharged amino acid; wherein X7 is Gly, a positive amino acid, a negatively charged amino acid or a polar uncharged amino acid; and wherein X8 is Gln, a positive amino acid, a negatively charged amino acid or a polar uncharged amino acid.

For example, disclosed are compositions comprising one or more of the peptide amphiphiles PA-C16-DS, PA-C16-CA, PA-C16-K5, PA-C16-K5-NO, PA-C16-YK, and PA-C16-YK5-NO, wherein the degrading sequence (DS) comprises the amino acid sequence GTAGLIGQ (SEQ ID NO:1) and wherein the DS comprises one or more amino acid substitutions.

In some aspects, the composition is a pharmaceutical composition. In some aspects, the disclosed compositions comprise one or more of the peptide amphiphiles and a pharmaceutically acceptable carrier.

In some aspects, the pharmaceutical compositions described herein can be sterile and contain any of the disclosed compositions for producing the desired response in a unit of weight or volume suitable for administration to a subject. In some aspects, the pharmaceutical compositions can contain suitable buffering agents, including, e.g., acetic acid in a salt: citric acid in a salt: boric acid in a salt; and phosphoric acid in a salt.

When administered, the disclosed compositions or pharmaceutical compositions can be administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term denotes an organic or inorganic ingredient, natural or synthetic, with which the active ingredient is combined to facilitate the application. The components of the pharmaceutical compositions also are capable of being co-mingled with the disclosed compositions, and with each other, in a manner such that there is no interaction which would substantially impair the desired pharmaceutical efficacy.

As used herein, the term “pharmaceutically acceptable carrier” refers to solvents, dispersion media, coatings, antibacterial, isotonic and absorption delaying agents, buffers, excipients, binders, lubricants, gels, surfactants that can be used as media for a pharmaceutically acceptable substance. The pharmaceutically acceptable carriers can be lipid-based or a polymer-based colloid. Examples of colloids include liposomes, hydrogels, microparticles, nanoparticles and micelles. The compositions can be formulated for administration by any of a variety of routes of administration, and can include one or more physiologically acceptable excipients, which can vary depending on the route of administration. Any of the compositions described herein can be administered in the form of a pharmaceutical composition.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions disclosed herein can be sterile and sterilized by conventional sterilization techniques developed for non-soluble biological material like collagens. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. The active ingredient can be any of the adipose or devitalized adipose tissues described herein in combination with one or more pharmaceutically acceptable carriers. As used herein “pharmaceutically acceptable” means molecules and compositions that do not produce or lead to an untoward reaction (i.e., adverse, negative or allergic reaction) when administered to a subject as intended (i.e., as appropriate).

In some aspects, administration of disclosed compositions or pharmaceutical compositions disclosed herein can be administered to mammals other than humans, e.g., for testing purposes or veterinary therapeutic purposes, can be carried out under substantially the same conditions as described above.

Disclosed are compositions comprising peptide amphiphile PA-C16-GTAGLIGK-YIGSR (SEQ ID NO:22) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence

(SEQ ID NO: 4)
Gly-Thr-Ala-Gly-Leu-Ile-Gly-Lys.

Disclosed are compositions comprising peptide amphiphile PA-C16-GTAGLIKK-YIGSR (SEQ ID NO:27) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence

(SEQ ID NO: 5)
Gly-Thr-Ala-Gly-Leu-Ile-Lys-Lys.

Disclosed are compositions comprising peptide amphiphile PA-C16-GTAGLKKK-YIGSR (SEQ ID NO:19) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence

(SEQ ID NO: 6)
Gly-Thr-Ala-Gly-Leu-Lys-Lys-Lys.

Disclosed are compositions comprising peptide amphiphile PA-C16-GTAGKKKK-YIGSR (SEQ ID NO:28) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence

(SEQ ID NO: 7)
Gly-Thr-Ala-Gly-Lys-Lys-Lys-Lys.

Disclosed are compositions comprising peptide amphiphile PA-C16-GTAKKKKK-YIGSR (SEQ ID NO:20) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Ala-Lys-Lys-Lys-Lys-Lys (SEQ ID NO:8) for improving PA solubility.

Disclosed are compositions comprising peptide amphiphile PA-C16-GTKKKKKK-YIGSR (SEQ ID NO:29) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Lys-Lys-Lys-Lys-Lys-Lys (SEQ ID NO:9) for improving PA solubility.

Disclosed are compositions comprising peptide amphiphile PA-C16-GKKKKKKK-YIGSR (SEQ ID NO:31) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Lys-Lys-Lys-Lys-Lys-Lys-Lys (SEQ ID NO: 10) for improving PA solubility.

Disclosed are compositions comprising peptide amphiphile PA-C16-KKKKKKKK-YIGSR (SEQ ID NO:30) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and a sequence for improving PA solubility Lys-Lys-Lys-Lys-Lys-Lys-Lys-Lys (SEQ ID NO:11).

Disclosed are compositions comprising peptide amphiphile PA-C16-GTAGLIGK-YIGSR (SEQ ID NO:22), wherein one or more of the Lys residues of the peptide amphiphile comprise a pendant amine group, wherein the pendant amine group can react with Nitric Oxide (NO) to form a diazeniumdiolate-modified peptide amphiphile (PA-C16-GTAGLIGK-YIGSR-NO (SEQ ID NO:22)).

Disclosed are compositions comprising peptide amphiphile PA-C16-GTAGLIKK-YIGSR (SEQ ID NO:27), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 10-NO: PA-C16-GTAGLIKK-YIGSR-NO (SEQ ID NO:27)).

Disclosed are compositions comprising peptide amphiphile PA-C16-GTAGLKKK-YIGSR (SEQ ID NO: 19), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 11-NO: PA-C16-GTAGLKKK-YIGSR-NO (SEQ ID NO:19)).

Disclosed are compositions comprising peptide amphiphile PA-C16-GTAGKKKK-YIGSR (SEQ ID NO:28), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 12-NO: PA-C16-GTAGKKKK-YIGSR-NO (SEQ ID NO:28)).

Disclosed are compositions comprising peptide amphiphile PA-C16-GTAKKKKK-YIGSR (SEQ ID NO:20), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 13-NO: PA-C16-GTAKKKKK-YIGSR-NO (SEQ ID NO:20)).

Disclosed are compositions comprising peptide amphiphile PA-C16-GTKKKKKK-YIGSR (SEQ ID NO:29), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 14-NO: PA-C16-GTKKKKKK-YIGSR-NO (SEQ ID NO:29)).

Disclosed are compositions comprising peptide amphiphile PA-C16-GKKKKKKK-YIGSR (SEQ ID NO:31), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 15-NO: PA-C16-GKKKKKKK-YIGSR-NO (SEQ ID NO:31)).

Disclosed are compositions comprising peptide amphiphile PA-C16-KKKKKKKK-YIGSR (SEQ ID NO:30), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 16-NO: PA-C16-KKKKKKKK-YIGSR-NO (SEQ ID NO:30)).

Disclosed are compositions comprising one or more of the peptide amphiphiles described herein and a pharmaceutically active agent. In some aspects, the pharmaceutically active agent is selected from a therapeutic drug for cardiovascular disease or an antibiotic

Disclosed are compositions comprising at least two peptide amphiphiles, a) a first peptide amphiphile, wherein the first peptide amphiphile is any of the peptide amphiphiles described herein; and b) a second peptide amphiphile, wherein the first and second peptides are present at a ratio of about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20.

In some aspects, the second peptide amphiphile can be PA-C16-K5 and thus comprises a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence of KKKKK (SEQ ID NO:3) and a degrading sequence (DS) comprising an amino acid sequence of GTAGLIGQ (SEQ ID NO:1).

In some aspects, the second peptide amphiphile can be PA-C16-K5-NO and thus comprises a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence of KKKKK (SEQ ID NO:3) and a degrading sequence (DS) comprising an amino acid sequence of GTAGLIGQ (SEQ ID NO: 1), wherein one or more of the Lys residues of the nitric oxide producing donor sequence comprises a pendant amine group, wherein the pendant amine group can react with Nitric Oxide (NO) to form a diazeniumdiolate-modified peptide amphiphile.

### 1. Peptide Amphiphile Plus Therapeutic

Disclosed are compositions comprising one or more peptide amphiphiles described herein, and a pharmaceutically active agent. In some aspects, the pharmaceutically active agent is selected from a therapeutic drug or a therapeutic drug releasing liposome for cardiovascular disease.

Disclosed herein are compositions comprising one or more peptide amphiphiles and a pharmaceutically active agent, wherein the pharmaceutically active agent is selected from a therapeutic drug or a therapeutic drug releasing liposome for cardiovascular disease. In some aspects, the therapeutic drug is selected from sirolimus, everolimus, paclitaxel, colchicine, statins, or miRNA. In some aspects, the therapeutic drug releasing liposome can be one or more of the therapeutic drug releasing liposomes described herein. In some aspects, a therapeutic drug is encapsulated in a liposome forming the therapeutic drug releasing liposome. For example, a therapeutic drug releasing liposome can comprise, or encapsulate, one or more of the peptide amphiphiles or compositions described throughout and a therapeutic drug. Examples of therapeutic drug releasing liposomes can be, but is not limited to, liposomal sirolimus, liposomal everolimus, liposomal colchicine, liposomal paclitaxel, or liposomal statin, or liposomal miRNA.

Disclosed herein are compositions and peptide amphiphiles that further comprise colchicine. The chemical name of the colchicine is N[5′,6,7,9-tetrahydro-1,2,3,10-tetratmethoxy 9-oxobenzo[a]heptalen-7-yl], (S)-acetamide; molecular formula: C22H25NO6; CAS number: 64-86-8. The chemical structure of colchicine is shown as follows:

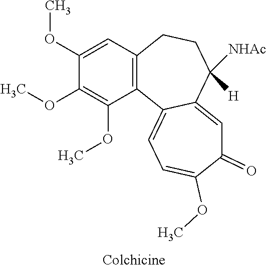 [SMILES: COc1cc2c(c(OC)c1OC)-c1ccc(OC)c(=O)cc1[C@@H](NC(C)=O)CC2]

Colchicine is a natural product used as an anti-inflammatory drug widely for treating symptomatic inflammatory diseases, such as gout. Colchicine is commonly extracted from two plants of the lily family, Colchicum autumnale and Gloriosa superba. Colchicine is a tricyclic alkaloid and has a molecular mass of 399.437. The active ingredient colchicine and its tablet formulation are listed in various national and international pharmacopeias such as the United States Pharmacopeia (USP).

Recently, colchicine has been studied for treating cardiovascular disease. For instance, In the Colchicine Cardiovascular Outcomes Trial (COLCOT) involving patients who had a myocardial infarction within 30 days before enrollment, the percentage of those who had the composite endpoint of cardiovascular death, resuscitated cardiac arrest, myocardial infarction, stroke, or urgent hospitalization for angina leading to coronary revascularization was lower among those who received 0.5 mg of colchicine once daily than among those who received placebo.

In an earlier trial of low-dose colchicine (LoDoCo) involving patients with chronic coronary disease, we found that the risk of acute cardiovascular events was lower among those who received 0.5 mg of colchicine once daily than those who did not receive colchicine. This was an open-label trial involving only 532 patients, and the results required confirmation. Accordingly, we conducted an investigator-initiated, randomized, controlled, double-blind, event-driven trial of low-dose colchicine (LoDoCo2) to determine whether 0.5 mg of colchicine once daily, as compared with placebo, prevents cardiovascular events in patients with chronic coronary disease.

Disclosed herein are compositions and peptide amphiphiles that further comprise sirolimus. Sirolimus is a white crystalline solid, of which the melting range is between 183° to 185° C. It is a lipophilic macrocyclic lactone structurally, soluble in most organic solvents, and virtually insoluble in water. Sirolimus is also known as rapamycin, the USAN-assigned generic name. Sirolimus is produced by a bacterium strain of Streptomyces hygroscopicus, known for its immunosuppressive and antiproliferative properties. Sirolimus consists of a 29-membered ring containing 4 trans double bonds, three of which are conjugated. The chemical structure of sirolimus is shown as follows:

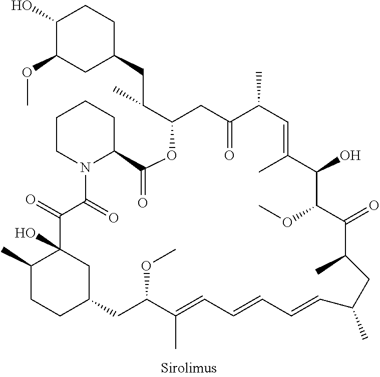 [SMILES: CO[C@H]1C[C@@H]2CC[C@@H](C)[C@@](O)(C2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Sirolimus is a potent inhibitor of S6Kl activation (a serine/threonine kinase) and a mediator of phosphoinositide 3-kinase (PI3K) signaling, which can form a gain-of-function complex with the FK506-binding protein (FKBP12) to bind and act as a specific allosteric inhibitor of the mechanistic target of rapamycin (mTOR) complex 1 (mTORC1). Because S6K and a eukaryotic translation initiation factor 4E (eIF4E)-binding protein 1 (4EBP1) are the main targets of mTORC1, mTOR inhibits 4EBP1 and activates S6K, thereby activating protein synthesis, ribosome biogenesis, nutrient transport, and lipid synthesis in response to nutrients, growth factors, and cellular energy. In addition, sirolimus can inhibit the activation of T and B cells.

Disclosed herein are compositions and peptide amphiphiles that further comprise a statin. Overwhelming evidence indicates that cardiovascular morbidity and mortality can be mitigated if the low-density lipoprotein cholesterol (LDL-C) is lowered. The frequently prescribed classic drugs for lowering cholesterol are statins, which are for primary and secondary prevention of CVD. The active part of statins is their modified 3,5-dihydroxyglutaric acid moiety, which is structurally similar to the endogenous substrate (hydroxymethylglutaryl-coenzyme A) HMG-COA, and the mevaldyl CoA transition state intermediate, thus, statins can inhibit HMG-COA reductase that can limit the rate of the cholesterol biosynthesis pathway. Commercially available statin drugs include atorvastatin, cerivastatin, fluvastatin, lovastatin, pitavastatin, pravastatin, rosuvastatin, and simvastatin. The structural differences of these statins are the rings attached to the active moiety. For instance, lovastatin, simvastatin, and pravastatin have a partially reduced naphthalene, while atorvastatin, Fluvastatin, and rosuvastatin have a pyrrole, an indole, and a pyrimidine, respectively. Below is the chemical structure of lovastatin.

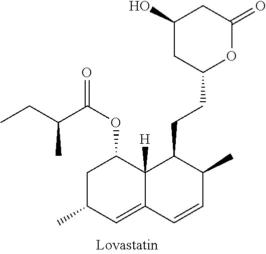 [SMILES: CC[C@H](C)C(=O)O[C@H]1C[C@@H](C)C=C2C=C[C@H](C)[C@H](CC[C@@H]3C[C@@H](O)CC(=O)O3)[C@H]21]

Statin therapy has demonstrated significant efficacy in lowering LDL-C levels. For instance, Statins only can decrease the 20-50% of the LDL-C level. In addition, It was reported that the LDL-C levels could be reduced to a low level (<40 mg/dL) when statin and PCSK9 inhibitors were used. Other Beneficial effects could include improved vascular endothelial function, cardiac regeneration, and re-endothelialization. Although statins provide lipid-lowering effects and reduce cardiovascular risk when administered systemically, they are often associated with systemic side effects, including muscle soreness, myopathy, liver damage, and increased risk of diabetes development. In addition, the more severe adverse effect may be associated with new-onset type 2, neurological and neurocognitive effects, hepatotoxicity, and renal toxicity. Local delivery of statins might provide greater statin concentration in target tissues while minimizing side effects.

Disclosed herein are compositions and peptide amphiphiles that further comprise one or more microRNAs (MiRNAs). In some aspects, one or more of the miRNAs provided in Tables 1-3 can be used in the disclosed compositions(SHOCK, 46(2), 122-131, (2016)). Table 1. Examples of miRNAs that can regulate M1 and M2 polarization through targeting various adaptor proteins and transcription factors that can be used in the disclosed compositions.

| MiRNAs | Phenotype | Targets | Function |
|---|---|---|---|
| miR-124 | M2 | STAT3 | TACE Inhibits production of pro- |
| | | | inflammatory cytokines |
| mrR-223 | M2 | STAT3 | Promotes anti-inflammatory response |
| miR-34a | M2 | Nothc1 | Inhibits production of pro-inflammatory |
| | | | cytokines |
| Let-7c | M2 | PAK1 | Inhibits activation of NF-kB pathway |
| miR-132 | M2 | AChE | Promotes cholinergic anti-inflammatory |
| | | | response |
| miR-146a | M2 | IRAK1, | Prevents activation of NF-kB |
| | | TRAF6 | |
| miR-125a | M2 | KLF4 | Promotes M2 polarization |

*PAK1 indicates p21-activated kinase 1.

Table 2. Examples of bone regeneration related miRNAs that can be used in the disclosed compositions. (Current Genomics. 16, 441-452 (2015)).

| MicroRNA | Cell | Target Gene |
|---|---|---|
| Let-7f | Human MSC | Axin2 |
| miRNA-15b | Human MSC | BMPR2 |
| miR-20a | Human MSC | PPARg, Bambi, Crim1 |
| miR-21 | Human MSC | Spry1 |
| miR-30c | Human MSC | CAMTA1*, CXCL12*, |
| | | ITGB1*, FLT1* |
| miR-96 | Human MSC | FABP4 |
| miR-130b | Human MSC | CAMTA1*, CD44*, |
| | | GDF6*, PDGFRA*, |
| | | COL9A3* |
| miR-199a | Human MSC | SOX9 |

*putative target gene

Table 3. Examples of vascular related miRNAs that can be used in the disclosed compositions. (Cardiovascular Research. 110, 6-22. (2016)).

| miRNA | Modulation | Effect |
|---|---|---|
| miR-122 | Antagomir | Cholesterol decrease |
| miR-145 | Lentiviral overexpression | Plaque size and fibrosis decrease |
| miR-21 | Antagomir | Neointimal proliferation decrease |
| miR-126 | Apoptotic bodies | Lesion size decrease, vascular |
| | | repair increase |

Disclosed are compositions comprising PA-C16-GTAGLIGK-YIGSR (SEQ ID NO:22), PA-C16-GTAGLIKK-YIGSR (SEQ ID NO:27), PA-C16-GTAGLKKK-YIGSR (SEQ ID NO: 19), PA-C16-GTAGKKKK-YIGSR (SEQ ID NO:28), PA-C16-GTAKKKKK-YIGSR (SEQ ID NO:20), PA-C16-GTKKKKKK-YIGSR (SEQ ID NO:29), PA-C16-GKKKKKKK-YIGSR (SEQ ID NO:31), PA-C16-KKKKKKKK-YIGSR (SEQ ID NO:30), PA-C16-GTAGLIGK-YIGSR-NO (SEQ ID NO:22), PA-C16-GTAGLIKK-YIGSR-NO (SEQ ID NO:27), PA-C16-GTAGLKKK-YIGSR-NO (SEQ ID NO:19), PA-C16-GTAGKKKK-YIGSR-NO (SEQ ID NO:28), PA-C16-GTAKKKKK-YIGSR-NO (SEQ ID NO:20), PA-C16-GTKKKKKK-YIGSR-NO (SEQ ID NO:29), PA-C16-GKKKKKKK-YIGSR-NO (SEQ ID NO:31), or PA-C16-KKKKKKKK-YIGSR-NO (SEQ ID NO:30) and a pharmaceutically active agent, such as a therapeutic drug or a therapeutic drug releasing liposome for cardiovascular disease.

Disclosed are compositions comprising two peptide amphiphiles and a therapeutic drug or a therapeutic drug releasing liposome for cardiovascular disease, a first peptide amphiphile, wherein the first peptide amphiphile is any of the peptide amphiphiles described herein; and a second peptide amphiphile; wherein the second peptide amphiphile (PA-C16-K5-NO) comprises a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence of KKKKK (SEQ ID NO:3) and a degrading sequence (DS) comprising an amino acid sequence of GTAGLIGQ (SEQ ID NO:1), wherein one or more of the Lys residues of the nitric oxide producing donor sequence comprises a pendant amine group, wherein the pendant amine group can react with Nitric Oxide (NO) to form a diazeniumdiolate-modified peptide amphiphile. In some aspects, the therapeutic drug can be sirolimus, everolimus, paclitaxel, colchicine, statins, or miRNA. In some aspects, the therapeutic drug can be a therapeutic drug releasing liposome such as, but not limited to, liposomal sirolimus, liposomal everolimus, liposomal paclitaxel, liposomal colchicine, liposomal statin, or liposomal statin.

Disclosed are compositions comprising two peptide amphiphiles and a therapeutic drug or a therapeutic drug releasing liposome for cardiovascular disease, wherein the composition comprises a first peptide amphiphile, wherein the first peptide amphiphile is any of the peptide amphiphiles described herein; and a second peptide amphiphile; wherein the second peptide amphiphile (PA-C16-K5) comprises a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence of KKKKK (SEQ ID NO:3) and a degrading sequence (DS) comprising an amino acid sequence of GTAGLIGQ (SEQ ID NO:1). In some aspects, the therapeutic drug can be sirolimus, everolimus, paclitaxel, colchicine, statins, or miRNA. In some aspects, the therapeutic drug can be a therapeutic drug releasing liposome such as, but not limited to, liposomal sirolimus, liposomal everolimus, liposomal paclitaxel, liposomal colchicine, liposomal statin, or liposomal statin.

In some aspects, the first and second peptides are present at a ratio of about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20.

### 2. Peptide Amphiphile Plus Antibiotic

Disclosed are compositions comprising one or more peptide amphiphiles described herein, and a pharmaceutically active agent. In some aspects, the pharmaceutically active agent is selected from an antibiotic or an antibiotic releasing liposome for preventing or treating infection.

Disclosed herein are compositions comprising one or more peptide amphiphiles and a pharmaceutically active agent, wherein the pharmaceutically active agent is selected from an antibiotic or an antibiotic releasing liposome for treating or preventing infection. In some aspects, the antibiotic is selected from gentamicin and vancomycin. In some aspects, the antibiotic releasing liposome is liposomal vancomycin or liposomal gentamicin.

Gentamicin, an aminoglycoside antibiotic, has been used to treat several gram-negative bacteria for infections, such as bone infection, pelvic inflammatory disease, and urinary tract infections. Commonly, gentamicin has been administrated through parenteral routes, such as systemic, topical, and ophthalmic formulations, including intramuscular and intravenous administration. The highly susceptible microorganisms to gentamicin are members of the Enterobacteriaceae family, such as Escherichia coli, Klebsiella pneumoniae, Serratia spp, and Enterobacter spp.), Pseudomonas aeruginosa, and some strains of Neisseria, Moraxella, and Haemophilus genera. The gentamicin is only effective for aerobic bacteria, as it enters the bacteria membrane through an oxygen-dependent active transport. After gentamicin passes through the membrane and stays in the cytoplasm, it leads to the formation of truncated or nonfunctional proteins by disturbing mRNA translation. Therefore, the gentamicin dosage used in the patient depends on the patient's weight. Although gentamicin is widely used in clinics, it still can result in inner van ear and kidney problems.

In contrast to gentamicin, vancomycin, a glycopeptide antibacterial, has been developed to treat severe gram-positive infections involving meticillin (methicillin)-resistant S. aureus (MRSA). Although vancomycin was regarded for its excellent efficacy more than its toxicity, recently, it was found that several types of bacteria, such as enterococci and later in staphylococci, showed resistance to vancomycin. Vancomycin is used to treat skin infections and bone and joint infections. Below are the chemical structures of gentamicin and vancomycin.

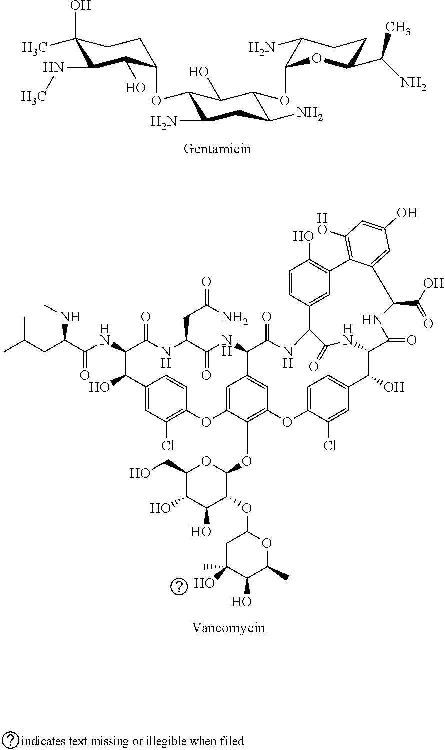

In some aspects, the antibiotic releasing liposome can be one or more of the antibiotic releasing liposomes described herein. In some aspects, an antibiotic is encapsulated in a liposome forming the antibiotic releasing liposome. For example, an antibiotic releasing liposome can comprise, or encapsulate, one or more of the peptide amphiphiles or compositions described throughout and an antibiotic.

Disclosed are compositions comprising two peptide amphiphiles and an antibiotic or an antibiotic releasing liposome, wherein a first peptide amphiphile is any of the peptide amphiphiles described herein; wherein a second peptide amphiphile (PA-C16-K5-NO) comprises a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence of KKKKK (SEQ ID NO:3) and a degrading sequence (DS) comprising an amino acid sequence of GTAGLIGQ (SEQ ID NO: 1), wherein one or more of the Lys residues of the nitric oxide producing donor sequence comprises a pendant amine group, wherein the pendant amine group can react with Nitric Oxide (NO) to form a diazeniumdiolate-modified peptide amphiphile. In some aspects, the antibiotic is vancomycin or gentamicin. In some aspects, the antibiotic can be an antibiotic releasing liposome such as, but not limited to, liposomal vancomycin or liposomal gentamicin.

Disclosed are compositions comprising two peptide amphiphiles and an antibiotic or an antibiotic releasing liposome, wherein a first peptide amphiphile is any of the peptide amphiphiles described herein; wherein a second peptide amphiphile (PA-C16-K5) comprises a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence of KKKKK (SEQ ID NO:3) and a degrading sequence (DS) comprising an amino acid sequence of GTAGLIGQ (SEQ ID NO:1). In some aspects, the antibiotic is vancomycin or gentamicin. In some aspects, the antibiotic can be an antibiotic releasing liposome such as, but not limited to, liposomal vancomycin or liposomal gentamicin.

In some aspects, the first and second peptides are present at a ratio of about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20.

### D. Liposomes

Disclosed are liposomes comprising one or more of the peptide amphiphiles or compositions disclosed throughout. For example, disclosed herein are liposomes comprising a peptide amphiphile comprising a hydrophilic peptide sequence and a hydrophobic tail, wherein the hydrophobic tail comprises a moiety having a substituted C16 alkyl chain, wherein the hydrophilic peptide sequence comprises a degrading sequence (DS), wherein the degrading sequence (DS) comprises the amino acid sequence GTAGLIGQ (SEQ ID NO:1) wherein the DS comprises one or more amino acid substitutions. In some aspects, the one or more amino acid substitutions can be any amino acid substitution.

Disclosed herein are liposomes comprising peptide amphiphiles comprising a hydrophilic peptide sequence and a hydrophobic tail, wherein the hydrophobic tail comprises a moiety having a substituted C16 alkyl chain, wherein the hydrophilic peptide sequence comprises a degrading sequence (DS) wherein the degrading sequence (DS) comprises an amino acid sequence comprising the sequence X₁X₂X₃X₄X₅X₆X₇X₈; SEQ ID NO: 18, wherein X1 is Gly, a positive amino acid, a negatively charged amino acid or a polar uncharged amino acid; wherein X2 is Thr, a positive amino acid, a negatively charged amino acid or a polar uncharged amino acid; wherein X3 is Ala, a positive amino acid, a negatively charged amino acid or a polar uncharged amino acid; wherein X4 is r Gly, a positive amino acid, a negatively charged amino acid or a polar uncharged amino acid; wherein X5 is Leu, a positive amino acid, a negatively charged amino acid or a polar uncharged amino acid; wherein X6 is Ile, a positive amino acid, a negatively charged amino acid or a polar uncharged amino acid; wherein X7 is Gly, a positive amino acid, a negatively charged amino acid or a polar uncharged amino acid; and wherein X8 is Gln, a positive amino acid, a negatively charged amino acid or a polar uncharged amino acid.

Disclosed are liposomes comprising peptide amphiphile PA-C16-GTAGLIGK-YIGSR (SEQ ID NO:22) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Ala-Gly-Leu-Ile-Gly-Lys (SEQ ID NO:4).

Disclosed are liposomes comprising peptide amphiphile PA-C16-GTAGLIKK-YIGSR (SEQ ID NO:27) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Ala-Gly-Leu-Ile-Lys-Lys (SEQ ID NO:5).

Disclosed are liposomes comprising peptide amphiphile PA-C16-GTAGLKKK-YIGSR (SEQ ID NO: 19) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Ala-Gly-Leu-Lys-Lys-Lys (SEQ ID NO:6).

Disclosed are liposomes comprising peptide amphiphile PA-C16-GTAGKKKK-YIGSR (SEQ ID NO:28) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Ala-Gly-Lys-Lys-Lys-Lys (SEQ ID NO:7).

Disclosed are liposomes comprising peptide amphiphile PA-C16-GTAKKKKK-YIGSR (SEQ ID NO:20) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Ala-Lys-Lys-Lys-Lys-Lys (SEQ ID NO:8) for improving PA solubility.

Disclosed are liposomes comprising peptide amphiphile PA-C16-GTKKKKKK-YIGSR (SEQ ID NO:29) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Lys-Lys-Lys-Lys-Lys-Lys (SEQ ID NO:9) for improving PA solubility.

Disclosed are liposomes comprising peptide amphiphile PA-C16-GKKKKKKK-YIGSR (SEQ ID NO:31) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Lys-Lys-Lys-Lys-Lys-Lys-Lys (SEQ ID NO:10) for improving PA solubility.

Disclosed are liposomes comprising peptide amphiphile PA-C16-KKKKKKKK-YIGSR (SEQ ID NO:30) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and a sequence for improving PA solubility Lys-Lys-Lys-Lys-Lys-Lys-Lys-Lys (SEQ ID NO: 11).

Disclosed are liposomes comprising peptide amphiphile PA-C16-GTAGLIGK-YIGSR PA-C16-GTAGLIGK-YIGSR, wherein one or more of the Lys residues of the peptide amphiphile comprise a pendant amine group, wherein the pendant amine group can react with Nitric Oxide (NO) to form a diazeniumdiolate-modified peptide amphiphile (PA-C16-GTAGLIGK-YIGSR-NO PA-C16-GTAGLIGK-YIGSR).

Disclosed are liposomes comprising peptide amphiphile PA-C16-GTAGLIKK-YIGSR (SEQ ID NO:27), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 10-NO: PA-C16-GTAGLIKK-YIGSR-NO (SEQ ID NO:27)).

Disclosed are liposomes comprising peptide amphiphile PA-C16-GTAGLKKK-YIGSR (SEQ ID NO: 19), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 11-NO: PA-C16-GTAGLKKK-YIGSR-NO (SEQ ID NO:19)).

Disclosed are liposomes comprising peptide amphiphile PA-C16-GTAGKKKK-YIGSR, wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 12-NO: PA-C16-GTAGKKKK-YIGSR-NO (SEQ ID NO:28)).

Disclosed are liposomes comprising peptide amphiphile PA-C16-GTAKKKKK-YIGSR (SEQ ID NO:20), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 13-NO: PA-C16-GTAKKKKK-YIGSR-NO (SEQ ID NO:20)).

Disclosed are liposomes comprising peptide amphiphile PA-C16-GTKKKKKK-YIGSR (SEQ ID NO:29), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 14-NO: PA-C16-GTKKKKKK-YIGSR-NO (SEQ ID NO:29)).

Disclosed are liposomes comprising peptide amphiphile PA-C16-GKKKKKKK-YIGSR (SEQ ID NO:31), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 15-NO: PA-C16-GKKKKKKK-YIGSR-NO (SEQ ID NO:31)).

Disclosed are liposomes comprising peptide amphiphile PA-C16-KKKKKKKK-YIGSR (SEQ ID NO:30), wherein one or more of the Lys residues of the peptide amphiphile comprise a pendant amine group, wherein the pendant amine group can react with Nitric Oxide (NO) to form a diazeniumdiolate-modified peptide amphiphile (peptide amphiphile 16-NO: PA-C16-KKKKKKKK-YIGSR-NO (SEQ ID NO:30))

Also disclosed are liposomes comprising one or more of the disclosed peptide amphiphiles in combination with a pharmaceutically active agent (e.g., therapeutic or antibiotic). For example, disclosed are liposomes comprising one or more peptide amphiphiles described herein and a therapeutic drug, wherein the one or more peptide amphiphiles comprise at least one peptide amphiphile comprising DS: PA-C16-DS, PA-C16-CA, PA-C16-K5. PA-C16-K5-NO. PA-C16-YK, or PA-C16-YK5-NO, wherein the DS of each comprises one or more amino acid substitutions.

In some aspects, the liposome further comprises cholesterol and lipids. In some aspects, the lipid is at least one of those selected from the group consisting of 1,2-distearoyl-snglycero-3-phosphocholine (DSPC). 1,2-dipalmitoyl-snglycero-3-phosphocholine (DPPC). 1,2-Distearoyl-sn-glycero-3-phosphoethanolamine-poly (ethylene glycol) (DSPE-PEG), or Dioleoyl-3-trimethylammonium propane (DOTAP). In some aspects, the lipids can be, but are not limited to, dioleoylphosphatidylglycerol (DOPG), dioleoylphosphatidylcholine (DOPC), dimyristoylphosphatidylcholine (DMPC), dioleoylphosphatidylserine (DOPS), palmitoyloleyolphosphatidylglycerol (POPG), dioleoylphosphatidylethanolamine (DOPE), dipalmitoyl phosphatidyl ethanolamine (DPPE), dimyristoylphosphoethanolamine (DMPE), distearoyl-phosphatidy 1-ethanolamine (DSPE), palmitoyloleoyl-phosphatidylethanolamine (POPE), palmitoyloleoylphosphatidylcholine (POPC), egg phosphatidylcholine (EPC), distearoylphosphatidylcholine (DSPC), dipalmitoylphosphatidylcholine (DPPC), dipalmitoylphosphatidylglycerol (DPPG), DSPG, palmitoyloleyolphosphatidylglycerol (POPG), palmitoyloleoyl-phosphatidylethanolamine (POPE). 1-stearoyl-2-oleoylphosphatidyethanolamine (SOPE), DSPE-(polyethylene glycol) PEG. DMPE-PEG. DPPE-PEG or DOPE-PEG.

In some aspects, the liposome has a diameter of about 40 nm to about 200 nm.

In some aspects, the pharmaceutically active agent is a therapeutic drug. In some aspects, the therapeutic drug is selected from sirolimus, everolimus, paclitaxel, colchicine, or statins. Thus, disclosed are liposomes comprising one or more of the peptide amphiphiles and one or more of sirolimus, everolimus, paclitaxel, colchicine, or statins. In some aspect, the liposome is a sirolimus releasing liposome, everolimus releasing liposome, paclitaxel,=releasing liposome, colchicine releasing liposome, or statin releasing liposome.

In some aspects, the pharmaceutically active agent is an antibiotic. In some aspects, the antibiotic is gentamicin or vancomycin. Thus, disclosed are liposomes comprising one or more of the peptide amphiphiles and one or more of gentamicin and vancomycin. In some aspect, the liposome is a gentamicin releasing liposome or vancomycin releasing liposome.

In some aspects, the liposome can be an anionic liposome, a cationic liposome, or a neutral liposome.

### E. Gels

Disclosed are compositions comprising two or more peptide amphiphiles and calcium chloride, wherein at least one peptide amphiphile comprises one or more molecules of NO, and wherein at least one peptide amphiphile is a peptide amphiphile described herein. In some aspects, the presence of the calcium chloride forms a gel. Therefore, in some aspects, the compositions can be a gel. Thus, also disclosed are gels comprising a composition comprising two or more peptide amphiphiles and calcium chloride, wherein at least one peptide amphiphile comprises one or more molecule of NO, and wherein at least one peptide amphiphile is a peptide amphiphile described herein.

In some aspects, the two or more peptide amphiphiles are different peptide amphiphiles.

In some aspects, a first peptide amphiphile is any peptide amphiphile described herein and a second peptide amphiphile is PA-C16-DS which comprises a hydrophilic peptide sequence and a hydrophobic tail, wherein the hydrophobic tail comprises a moiety having substituted C16 alkyl chain, wherein the hydrophilic peptide sequence comprises a degrading sequence (DS) comprising an amino acid sequence GTAGLIGQ (SEQ ID NO:1), wherein the degradation sequence comprises a matrix metalloprotease (MMP) specific cleavage site.

In some aspects, the disclosed gels can comprise a peptide amphiphile and at least one unmodified peptide amphiphiles, wherein the peptide amphiphile is any peptide amphiphile described herein and a first unmodified peptide amphiphile is PA-C16-K5-NO which comprises a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence of KKKKK (SEQ ID NO:3) and a degrading sequence (DS) comprising an amino acid sequence of GTAGLIGQ (SEQ ID NO:1), wherein one or more of the Lys residues of the nitric oxide producing donor sequence comprises a pendant amine group, wherein the pendant amine group can react with Nitric Oxide (NO) to form a diazeniumdiolate-modified peptide amphiphile, and a second unmodified peptide amphiphile is PA-C16-DS which comprises a hydrophilic peptide sequence and a hydrophobic tail, wherein the hydrophobic tail comprises a moiety having substituted C16 alkyl chain, wherein the hydrophilic peptide sequence comprises a degrading sequence (DS) comprising an amino acid sequence GTAGLIGQ (SEQ ID NO:1), wherein the degradation sequence comprises a matrix metalloprotease (MMP) specific cleavage site. In some aspects, an “unmodified peptide” as used herein can mean any of the disclosed peptide amphiphiles without a modified DS.

In some aspects, the compositions, including the gels, further comprise a pharmaceutically active agent. In some aspects, the pharmaceutically active agent can be encapsulated in a liposome forming a therapeutic drug releasing liposome or antibiotic releasing liposome. In some aspects, the pharmaceutically active agent is selected from a therapeutic drug or a therapeutic drug releasing liposome for cardiovascular disease. In some aspects, the therapeutic drug is selected from sirolimus, everolimus, paclitaxel, colchicine, statins, or miRNA. In some aspects, the pharmaceutically active agent is selected from an antibiotic or an antibiotic releasing liposome for treating infection. In some aspects, the antibiotic is selected from gentamicin and vancomycin.

Thus, for example, disclosed are gels comprising two or more peptide amphiphiles, a pharmaceutically active agent, and calcium chloride, wherein the first peptide is PA-C16-YK5-NO, and the second peptide is PA-C16-DS, and wherein the pharmaceutically active agent is sirolimus, everolimus, paclitaxel, colchicine, or statins.

In some aspects, the gels described herein comprise two or more peptide amphiphiles and calcium chloride but no pharmaceutically active agent. In some aspects, the gels disclosed herein can be used to treat arteriovenous fistula failure.

In some aspects, the gels described herein comprise two or more peptide amphiphiles, calcium chloride, and a pharmaceutically active agent. In some aspects, these gels can be used to treat a disease that the pharmaceutically active agent is specific for. For example, cardiovascular disease can be treated if sirolimus, everolimus, paclitaxel, colchicine, statins, or a liposome releasing one or more of sirolimus, everolimus, paclitaxel, colchicine, or statins are the pharmaceutically active agent.

Disclosed are gels comprising one or more of the peptides described herein, one or more of the compositions described herein, or one or more of the liposomes described herein.

### F. Medical Devices

Disclosed are medical devices coated with one or more of the peptide amphiphiles, compositions, liposomes, or gels described herein. For example, disclosed are medical devices coated with a composition comprising one or more of the peptide amphiphiles and a pharmaceutically active agent. In some aspects, a pharmaceutically active agent is selected from a therapeutic drug or a therapeutic drug releasing liposome for cardiovascular disease. In some aspects, the therapeutic drug is selected from sirolimus, everolimus, paclitaxel, colchicine, statins, or miRNA. In some aspects, the therapeutic drug releasing liposome can be one or more of the therapeutic drug releasing liposomes described herein. In some aspects, a therapeutic drug is encapsulated in a liposome forming the therapeutic drug releasing liposome. For example, a therapeutic drug releasing liposome can comprise, or encapsulate, one or more of the peptide amphiphiles or compositions described throughout and a therapeutic drug. Examples of therapeutic drug releasing liposomes can be, but is not limited to, liposomal sirolimus, liposomal everolimus, liposomal colchicine, or liposomal statin, or liposomal miRNA. In some aspects, a pharmaceutically active agent is selected from an antibiotic or an antibiotic releasing liposome. In some aspects, the antibiotic is selected from gentamicin and vancomycin. In some aspects, the antibiotic releasing liposome is liposomal vancomycin or liposomal gentamicin.

In some aspects, the medical device is a cardiovascular medical device. For example, a cardiovascular medical device can be a vascular stent, vascular graft, catheter, pacemaker, or heart valve.

In some aspects, the medical device can be, but is not limited to, coronary stents, drug eluting balloons, brain aneurysm coils, brain flow diverters, total knee arthroplasty, total disc arthroplasty, total hip arthroplasty, or percutaneous osseointegrated prostheses (POP).

In some aspects, a medical device can be any device known or identified for use inside a subject's body. Preferably, the medical device is one that is inserted into the cardiovascular system. In addition, the medical device can comprise any material suitable for use as a surgical implant.

Percutaneous coronary intervention has been used to treat atherosclerotic plaques clinically, which sometimes lead to barotrauma with injury to the endothelium and the vessel wall. Restenosis happens after standard balloon angioplasty, primarily due to elastic recoil of the vessel wall. Restenosis detected using coronary angiography is commonly defined as a binary event with re-narrowing of >50% of the vessel diameter. Although BMS demonstrated effectiveness in treating coronary artery disease, in-stent restenosis represented the central issue of BMS. Moreover, BMS implantation is associated with a high (nearly 40%) recurrence of binary restenosis. In addition, it has some critical long-term disadvantages, including limiting normal vasomotion and adaptive arterial remodeling, preclusion of bypass surgery, and persistent, chronic foreign body reaction that elicits inflammation from long-term metallic implants.

Thus, to address the issue of bare-metal stent (BMS), another type of stents, known as drug-eluting stents, have been developed. Drug-eluting stents (DES) can elute anti-proliferative agents, reducing in-stent restenosis. Current anti-proliferaive agents include everolimus, sirolimus, and biolimus. Current generation DES includes both durable and biodegradable polymer-coated stents, and the lifelong presence of durable polymer is reported to be related to chronic inflammation and neoatherosclerosis. Examples of drug-eluting stents include sirolimus-eluting stents such as Orsiro. COMBO Plus, and Ultimaster Tansei from BIOTRONIK. OrbusNeich, and Terumo. Despite DES demonstrating significant efficacy in suppressing restenosis; however, sirolimus released from DES is associated with a higher risk of endothelial apoptosis and incomplete re-endothelialization. Moreover, recent studies have revealed that the clinically used dose of sirolimus and its analogs cause serious adverse effects, including 1) damage to the endothelium, and 2) inflammation responses to the polymer coating that delivers sirolimus, and 3) the persistent risk of late thrombosis. Thus, it is imperative to develop effective approaches, such as the delivery of other agents along with sirolimus, to limit the adverse effects of sirolimus or its analogs at their currently used dose.

In addition to anti-proliferative agents, one option to avoid implanting multiple metallic layers of stents into ISR could be bioresorbable scaffolds (BRS), which, compared with DEB, can achieve more significant acute gain and prevent restenotic tissue prolapse and cover any edge dissection. So far, the BRS has been made primarily from PLLA, followed by magnesium and iron. Because PLLA has lower tensile and mechanical strength, stiffness, and ductility than steel or CoCr, scaffolds made from PLLA have thicker struts and a wider strut profile than metallic stents to improve tensile and radial strength. The most typical example of BRS is the Abbott Bioresorbable Vascular Scaffold (BVS) developed by Abbott Vascular, consisting of a poly d,llactide (PDLLA) backbone with a coating in a 1:1 ratio with everolimus. However, in 2017, based on a meta-analysis of 2-year outcomes of seven randomized clinical trials. Abbott Bioresorbable Vascular Scaffold was shown to have significantly higher rates of target-vessel myocardial infarction and stent thrombosis than DES, the everolimus-eluting, cobalt-chromium metallic stent. Thus. Abbott Bioresorbable Vascular Scaffold was withdrawn from the market. In addition to Abbott Bioresorbable Vascular Scaffold (BVS), other BVSs are under investigation, such as DESolve Nx from Elixir Medical. Fantom from REVA Medical, Magmaris from Biotronik, and XINSORB from HuaAn Biotechnology Coronary stents were developed to prevent the arterial recoiling process and acute closure due to arterial dissection which limited the effectiveness of coronary balloon angioplasty. Importantly, BVS can be coated with various drugs to functionalize as DES.

Disclosed herein are medical devices, such as a stents thst can comprise titanium alloy. In an aspect, the medical device can comprise cobalt-cromium. The medical device can comprise nickel-titanium. In an aspect, the medical device can comprise a biodegradable polymer. In some aspects, the medical device is a vascular stent. In some aspects, the stent is a drug-eluting stent. For example, the stent can be a sirolimus-eluting stent or a paclitaxel-eluting stent.

The skilled person in the field can appreciate additional medical devices for use with the disclosed NO-releasing nanomaterials and a pharmaceutically active agent. Preferably, the medical device administered to a tissue or organ of the body usually comprises a natural endothelium. For example, in some aspects, the medical device is a vascular graft in some aspects. In some aspects, the medical device is a catheter. In some aspects, the medical device is a pacemaker. In some aspects, the medical device is a heart valve.

### G. Methods

Disclosed are methods of preventing infection in a subject comprising administering to the subject a therapeutically effective amount of one or more of the peptide amphiphiles, mixtures of the peptide amphiphiles, compositions, liposomes, gels, or medical devices described herein to a subject in need thereof.

Disclosed are methods of treating a subject in need thereof comprising administering one or more of the medical devices described herein to a subject during osseointegration.

Disclosed are methods of increasing vascularization in a subject comprising administering one or more of the gels or the medical devices described herein to a subject in need thereof.

Disclosed are methods of treating inflammation in a subject comprising administering one or more of the gels or the medical devices described herein to a subject in need thereof.

Disclosed are methods of treating atherosclerosis in a subject comprising administering to the subject a therapeutically effective amount of one or more of the compositions, liposomes, gels, or medical devices described herein to a subject in need thereof.

Disclosed are methods of treating cardiovascular disease in a subject comprising administering to the subject a therapeutically effective amount of one or more of the compositions, liposomes, gels, or medical devices described herein to a subject in need thereof. For example, disclosed are methods of treating cardiovascular disease in a subject comprising administering to the subject a therapeutically effective amount of a composition comprising a NO-releasing peptide amphiphile with one or more substituted amino acid described herein, a drug including sirolimus and/or colchicine, and a peptide amphiphile PA-C16-DS that can form a gel when CaCl₂ is applied.

Disclosed are methods of treating arteriovenous fistula failure in a subject, comprising administering to the subject a therapeutically effective amount of one or more of the peptide amphiphiles, compositions, liposomes, gels, or medical device described herein to a subject in need thereof. In some aspects, the gel comprises a peptide amphiphile and a first unmodified peptide amphiphile and a second unmodified peptide amphiphile, wherein the peptide amphiphile is one or more of those disclosed herein, wherein the first unmodified peptide amphiphile is the peptide PA-C16-K5-NO, wherein the second unmodified peptide amphiphile (PA-C16-DS) comprises a hydrophilic peptide sequence and a hydrophobic tail, wherein the hydrophobic tail comprises a moiety having a substituted C16 alkyl chain, wherein the hydrophilic peptide sequence comprises a degrading sequence (DS) comprising an amino acid sequence GTAGLIGQ (SEQ ID NO:1), wherein the degradation sequence comprises a matrix metalloprotease (MMP) specific cleavage site. In some aspects, treating arteriovenous fistula failure in a subject can be performed by administering to the subject a therapeutically effective amount of one or more of the peptide amphiphiles, compositions, liposomes, gels, or medical device, wherein the peptide amphiphile comprises PA-C16-DS. PA-C16-CA, PA-C16-K5. PA-C16-K5-NO. PA-C16-YK, and PA-C16-YK5-NO, wherein the degrading sequence (DS) comprises the amino acid sequence GTAGLIGQ (SEQ ID NO:1) and wherein the DS comprises one or more amino acid substitutions.

In some aspects, the gel comprises a peptide amphiphile and a first peptide amphiphile and a second peptide amphiphile, wherein the peptide amphiphile is one or more of those disclosed herein, wherein the first peptide amphiphile is the peptide PA-C16-K5-NO, wherein the second peptide amphiphile (PA-C16-DS) comprises a hydrophilic peptide sequence and a hydrophobic tail, wherein the hydrophobic tail comprises a moiety having a substituted C16 alkyl chain, wherein the hydrophilic peptide sequence comprises a degrading sequence (DS) comprising an amino acid sequence GTAGLIGQ (SEQ ID NO:1), wherein the degradation sequence comprises a matrix metalloprotease (MMP) specific cleavage site.

Disclosed are methods of treating arteriovenous fistula failure in a subject, comprising administering to the subject a therapeutically effective amount of gel described herein to a subject in need thereof. In some respects, the gel comprises a first unmodified peptide amphiphile and a second peptide amphiphile, wherein the first unmodified peptide amphiphile is the peptide PA-C16-CA, wherein the second peptide amphiphile is PA-C16-GTAGLKKK-YIGSR-NO (SEQ ID NO:19), wherein one or more of the Lys residues of the peptide amphiphile comprise a pendant amine group, wherein the pendant amine group can react with Nitric Oxide (NO) to form a diazeniumdiolate-modified peptide amphiphiles. In some aspects, the gel comprises a first unmodified peptide amphiphile and a second peptide amphiphile, wherein the first unmodified peptide amphiphile is the peptide PA-C16-K5-NO, wherein the second peptide amphiphile is PA-C16-GTAGLKKK-YIGSR (SEQ ID NO:19) which comprises a hydrophilic peptide sequence and a hydrophobic tail, wherein the hydrophobic tail comprises a moiety having optionally substituted C16 alkyl chain; wherein the hydrophilic peptide sequence comprises an amino acid sequence Tyr-Ile-Gly-Ser-Arg (YIGSR; SEQ ID NO:2) and an amino acid sequence Gly-Thr-Ala-Gly-Leu-Lys-Lys-Lys (SEQ ID NO:6), amphiphiles.

### 1. Use of Disclosed Medical Devices

Disclosed are medical devices coated with the one or more of the disclosed compositions comprising NO-releasing nanomaterials and therapeutic drugs. Any of the disclosed medical devices can be used to treat subjects in need thereof.

Disclosed are methods of implanting (e.g. administering) the disclosed coated medical devices into a subject. Thus, in one aspect, the disclosed methods can comprise the steps of implanting a composition comprising a medical device (e.g., stent, vascular graft, catheter, pacemaker, or heart valve) coated with NO-releasing nanomaterials and a pharmaceutical agent into a subject. In some aspects, a medical device is coated with NO-releasing nanomaterials and a pharmaceutical agent prior to implanting into a subject. In a further aspect, a method can comprise the step of coating NO-releasing nanomaterials and a pharmaceutical agent onto a medical device after implantation into a subject.

### 2. Use of Disclosed Gels

As disclosed here, the composition disclosed herein can be in the form of a gel and the gel can be used to treat a subject in need thereof. In some aspects, the NO-releasing gel can be delivered to a subject through injection. In some aspects, the composition comprises a NO-releasing peptide amphiphile and peptide amphiphile PA-C16-DS that can form a NO-releasing gel when CaCl₂) is applied. In one example, a NO-releasing gel comprising NO-releasing peptide amphiphile (PA-C16-YK5-NO, wherein the DS comprises one or more amino acid substitutions) and peptide amphiphile PA-C16-DS and CaCl₂) was applied perivascularly at the arteriovenous anastomosis immediately following rat AVF creation. The NO-releasing nanomatrix gel inhibited intimal hyperplasia formation (more than 70% reduction), as well as improved vascular outward remodeling (increased vein diameter) and hemodynamic adaptation (lower wall shear stress approaching the preoperative level and less vorticity).

In addition, a method comprises the step of making the gelled macromolecules composed of NO-releasing peptide amphiphile, drugs, peptide amphiphile PA-C16-DS, and CaCl₂) before administration.

For peptide amphiphile PA-C16-DS, at neutral pH, the net negative charge can prevent the peptide amphiphile from self-assembling into gel structure; instead, the peptide amphiphiles remain amorphous. However, upon adding a high concentration of Ca 2, the repulsive negative charges can be eliminated, and the peptide amphiphile undergoes physical cross-linking to provide the gelled macrostructure. The methods for administering NO-releasing gel into a subject include but are not limited to, oral administration, transdermal administration, administration by inhalation, nasal administration, topical administration, intravaginal administration, ophthalmic administration, intraaural administration, intracerebral administration, rectal administration, and parenteral administration, including injectables such as intravenous administration, intra-arterial administration, intramuscular administration. In addition to treating AVF maturation failure, the NO-releasing gel is also promising for treating cardiovascular disease.

### 3. Cardiovascular Disease Treatment

Cardiovascular disease (CVD) is the leading cause of death in the United States. The most frequent CVD cause is atherosclerosis, which is an inflammatory disease. Current therapeutic options for treating cardiovascular atherosclerosis mostly are cholesterol-lowering drugs, such as statins. In addition, stents are the most implanted devices used to treat cardiovascular diseases. However, bare-metal stent (BMS) use remains limited by high rates of in-stent restenosis resulting from neointimal proliferation in response to vessel injury during stent deployment. To address this, drug-eluting stents (DES) coated with anti-proliferative agents such as sirolimus or sirolimus analogs have been developed to reduce restenosis by targeting the biochemical pathways of neointimal hyperplasia. However, sirolimus released from DES is associated with a higher risk of endothelial apoptosis and incomplete re-endothelialization. Moreover, recent studies have revealed that the clinically used dose of sirolimus and its analogs cause serious adverse effects, including 1) damage to the endothelium, 2) inflammation responses to the polymer coating that delivers sirolimus, and 3) the persistent risk of late thrombosis. Thus, it is imperative to develop effective approaches to limit the adverse effects of sirolimus or its analogs at their currently used dose.

Inflammation resolution has been studied as a potential novel approach for treating atherosclerosis. Colchicine, is an alkaloid extracted from Colchicum autumnale, Gloriosa superba, and other plants, with a chemical name (−)-N-[(7S, 12a5)-1,2,3,10-tetramethoxy-9-oxo-5,6,7,9-tetrahydrobenzo[a]heptalen-7-yl]-acetamide. It is a microtubule-disrupting agent clinically used to treat acute flares of gouty arthritis, familial Mediterranean fever (FMF), and Behçet's disease. In recent years, colchicine has been investigated as a potential therapeutic for treating CVDs, due to its potent anti-inflammatory properties. A recent meta-analysis of four major randomized controlled trials demonstrated that the use of colchicine was associated with a significant reduction in the primary composite endpoint of cardiovascular mortality, myocardial infarction, ischemic stroke, and urgent revascularization. Moreover, evidence from recent clinic trial studies demonstrated that low-dose colchicine could decrease the risk of acute cardiovascular events in patients with chronic coronary disease. Colchicine is therapeutic to improve outcomes in coronary atherosclerosis, pericarditis, atrial fibrillation and heart failure.

Another potential class of drugs for local delivery to reduce inflammation are statins. Statins provide lipid-lowering effects and reduce cardiovascular risk when administered systemically. However, they are often associated with systemic side effects, including muscle soreness, myopathy, liver damage and increased risk of diabetes development. Local delivery of statins might provide greater stain concentration in target tissues while minimizing side effects. Beneficial effects could include improved vascular endothelial function, cardiac regeneration, and improved re-endothelialization

In sum, the desire to reduce the delivered dose of sirolimus used as well as the potential benefits of NO, colchicine, and statins for CVD therapy, resulted in the disclosed co-delivery of NO with sirolimus, colchicine or statin for improving the treatment of CVD. This can improve efficacy and decrease potential adverse effects from delivering sirolimus or other medications.

As a solution to the problems associated with treating cardiovascular disease, disclosed are methods of treating a subject comprising administering to the subject a therapeutically effective amount of one or more of the peptide amphiphiles, mixtures of the peptide amphiphiles, compositions, liposomes, gels, or medical devices described herein to a subject in need thereof.

### 4. Amputation Treatment

Approximately 1.6 million people live with an amputation within the US, and amputation cases are expected to rise to approximately 3.6 million by 2050. Amputation is recommended for many reasons in the overall population, including peripheral artery disease (leading cause), severe trauma, tumor, or infection. Major lower limb amputations are much more frequent, with an estimated prevalence of nearly 40% of all limb loss. Transtibial is the most prevalent lower limb amputation at 52%, followed by transfemoral at 25%. The outcome of limb loss often leads to a decline in physical, social, and financial wellbeing. Returning to normal life and/or continuing pre-injury activities is challenged by conventional prosthetic techniques. However, current socket technology is associated with discomfort and functional limitations, especially in patients with short residual limbs or multiple limb loss. The conventional prosthetic sockets technology is unable to adapt to the dynamic residual limb as it atrophies over time and swells with heat or weight gain. In addition, socket prosthetics are prone to skin irritation, pain, and problems with prosthetic fixation. The issues of comfort and fit associated with the socket reduce the quality of life and mobility of the patient.

Percutaneous osseointegrated prostheses (POP) are a promising development for the limb-prosthesis interface involving the direct skeletal attachment of the prosthetic device. There are many variations of the implant system, the anchoring portion of the POP, including osseointegrated prostheses for the rehabilitation of amputees (OPRA), integral leg prosthesis (ILP), osseointegrated prosthetic limb (OPL), intraosseous transcutaneous amputation prosthesis (ITAP), keep walking advanced, and POP. To date, OPRA, ILP, and OPL are the only commercially available implant systems. POP improves donning and doffing, comfort, fit, skin irritation, range of motion, and osseo-proprioception over traditional socket prosthetics. Alongside the promising benefits of POP, significant risks are present at the bone-implant interface, including superficial and deep infection, inflammation, insufficient osseointegration, lack of vascularization, and implant loosening: 1) Infection: In screw implants, infection in soft tissue (Grade 1-2) occurs in 28% of cases and in bone in 5-13% (Grade 3). The introduction of bacteria to the implant site leads to the formation of an antibiotic-resistant biofilm and/or invasion into osteoblasts reducing bone tissue formation. 2) Inflammation: A study retrospectively assessing femoral implant patients found a 10-year cumulative risk for implant-associated osteomyelitis of 20% and a subsequent extraction in 9% of cases. Infection and biomaterial rejection are significant causes of inflammation at the implant and/or soft tissue site. It is critical for POP devices to avoid implementing immunogenic biomaterials to avoid rejection. To date, attempts to confront inflammation proactively have been confined to the aseptic operative technique. 3) Vascularization: Vascularization at the implant site fosters an environment rich in nutrients, biomolecules, and cellularity to encourage bone development, regeneration, and remodeling. 4) Osseointegration: Reports indicate the incidence of screw implant loosening within the bone varied from 3-23% of reported cases. Titanium has been implemented in the majority of modern POP designs due to its strength and biocompatibility. However, long-term studies have shown that a layer of fibrous tissue forms between the implant and bone. To further improve implant surface characteristics, hydroxyapatite is often coated onto the implant surface to form a stronger bond and quicker fixation with bone. However, plasma spraying hydroxyapatite onto the implant surface requires a high temperature leading to an elevated bio-dissolution rate.

Although there have been efforts to improve antibacterial effects or osseointegration of POP, there are no studies to improve multiple aspects, including angiogenesis and inflammation. The disclosed peptide Amphiphiles and uses thereof overcome current issues in POP by ameliorating infection, suppressing inflammation, and enhancing vascularization and osseointegration.

The direct incorporation of NO into POP intended for osseointegration or bone healing has never been attempted. However, NO plays a role in promoting angiogenesis via recruiting perivascular and endothelial cells. NO has also been shown to affect vascular endothelial growth factor release during angiogenesis occurring in bone remodeling. In addition, NO promotes the synthesis of cGMP, thereby activating downstream pathways, including cGMP-dependent kinase II (cGKII), which is known to play an essential role in endochondral bone formation. Since healing of a bone fracture undergoes endochondral ossification, using the NO-releasing multifunctional nanomatrix can promote the osseointegration of POP.

For antibacterial effects, NO is known to act as a signaling molecule in promoting the detachment of biofilms but does not affect host cell viability. NO can protect host cells from oxidative damage by terminating lipid peroxidation reactions. Recently, concentration-dependent antibacterial effects of the NO-releasing Nanomatrix were demonstrated. For anti-inflammatory effects of NO, NO synthase systems are associated with macrophages' wound healing processes. The NO-releasing multifunctional nanomatrix significantly decreased monocyte adhesion and pro-inflammatory gene expression.

Therefore, to tackle the critical issues of infection, osseointegration, vascularization, and inflammation of current POP, the disclosed compositions comprising a multifunctional NO and low dose of antibiotics have been used. These compositions can be used for improved osseointegration of prosthetics and other orthopedic and dental implants to help promote healing and prevent infection.

As a solution to the problems associated with amputation, disclosed are methods of treating a subject comprising administering to the subject a therapeutically effective amount of one or more of the peptide amphiphiles, mixtures of the peptide amphiphiles, compositions, liposomes, gels, or medical devices described herein to a subject in need thereof.

### 5. Arteriovenous Fistulas Treatment

A working vascular access is the “lifeline” for hemodialysis patients. Over 700,000 patients with End-Stage Renal Disease (ESRD), and 70% of these patients utilize hemodialysis as their kidney replacement modality of choicel. A functioning and durable vascular access provides the conduit to achieve consistent dialysis therapy for the hemodialysis patient. Arteriovenous fistulas (AVFs), created by a direct anastomosis between a native artery and vein are the preferred type of vascular access because AVFs have substantially lower thrombosis rates, infection and healthcare-related expenditures if they mature successfully for dialysis. However, AVF maturation failure remains a significant cause of morbidity, mortality, and hospitalization among hemodialysis patients. Results from a multicenter randomized clinical trial in the United States reported that 60% of AVFs created in hemodialysis patients fail to mature for dialysis use. The annual costs of treating vascular access dysfunction are over two billion U.S. dollars, mainly because the high proportion of non-maturing AVFs result in frequent interventions to promote maturation and long-term dialysis catheter use. Despite the magnitude of AVF maturation failure, we presently have no effective therapies to prevent or treat this clinical problem. Thus, identifying therapeutic targets and developing local novel therapies to reduce AVF maturation failure in hemodialysis patients represents an unmet clinical need.

The endothelium plays an essential role in regulating AVF development. In addition to the benefits of NO for vascular remodeling discussed previously, endothelial-derived NO is a key vasodilator and signaling molecule in vascular remodeling, and it has also been shown to inhibit intimal hyperplasia in arterial injury models. Moreover, the overexpression of NOS: reduced initial hyperplasia development in AVFs supported the hypothesis that increasing and sustaining local NO bioavailability—concurrent with AVF creation—can inhibit AVF maturation failure. Thus described herein are methods of administering NO, via peptide Amphiphiles, to the AVF. Importantly, it was demonstrated that the NO-releasing gel was successfully applied at the venous anastomosis of the rat AVF and reduced both venous intimal hyperplasia and a pro-inflammatory mediator and other markers of inflammation compared to the control nanomatrix gel (without NO) treated group by histological, biological analysis, and transcriptomics analysis. Thus, described herein is a local delivery of NO to address AVF maturation failure.

As a solution to the problems associated with treating AVF, disclosed are methods of treating a subject comprising administering to the subject a therapeutically effective amount of one or more of the peptide amphiphiles, mixtures of the peptide amphiphiles, compositions, liposomes, gels, or medical devices described herein to a subject in need thereof.

### H. Methods of Making

The compositions disclosed herein and the compositions necessary to perform the disclosed methods can be made using any method known to those of skill in the art for that particular reagent or compound unless otherwise explicitly noted.

In an aspect, disclosed is a method of producing the disclosed peptide amphiphiles, such as SEQ ID NO:4 to SEQ ID NO:11, by linking two or more peptides or polypeptides together by protein chemistry techniques and solid peptide synthesis (SPPS). Particularly, peptides or polypeptides can be chemically synthesized using currently available laboratory equipment, either Fmoc (9)-fluorenylmethyloxycarbonyl) or Boc (tert-butyloxycarbonoyl) chemistry. (Applied Biosystems. Inc., Foster City, Calif.). One skilled in the art can readily appreciate that a peptide or polypeptide corresponding to the disclosed proteins, for example, can be synthesized by standard chemical reactions. The solid-phase peptide synthesis (SPPS) is traditionally carried out in the C→N direction. Most peptides are synthesized as C-terminal acids or amides. By peptide condensation reactions, these two fragments can be covalently joined via a peptide bond at their carboxyl and amino termini, respectively, to form an antibody or fragment thereof. Successful SPPS depends on the solid support, linker (between the solid support and the synthesized peptide), appropriately protected amino acids, coupling methodology, and protocol for cleaving the peptide from the solid support. The solid support often refers to commensally available resin. The term coupling refers to forming a peptide bond between two adjacent amino acids. The typical examples of in situ coupling reagents are N,N′-dicyclohexylcarbodiimide (DCC), and the related N,N′-diisopropylcarbodiimide (Rich and Singh, 1979). The generality of carbodiimide-mediated couplings is extended significantly. The use of either 1-hydroxy benzotriazole (HOBt) or 1-hydroxy-7-azabenzotriazole (HOAt) as an additive, either of which accelerates carbodiimide-mediated couplings, suppresses racemization, and inhibits dehydration of the carboxamide side chains of Asn and Gln to the corresponding nitriles. In addition to carborbodiimides, protocols involves chemicals, such as, hexafluorophosphate (PyAOP), O-benzotriazol-1-yl-N,N,N′,N′-tetramethyluronium hexafluorophosphate (HBTU), O-(7-azabenzotriazol-1-yl)-N,N,N′N′-tetramethyluronium hexafluorophosphate (HATU), O-(6-Chlorobenzotriazol-1-yl)-N, N,N′,N′-tetramethyluronium hexafluorophosphate (HCTU), leads to more rapid coupling kinetics than these obtained with carbodiimides.

For resin cleavage, in some cases, a peptide or polypeptide can be synthesized and not cleaved from its synthesis resin, whereas the other fragment of a peptide or protein can be synthesized and subsequently cleaved from the resin, thereby exposing a terminal group that is functionally blocked on the other fragment.

It is contemplated that a disclosed peptide amphiphiles can be prepared by attachment of a hydrophobic moiety via conventional synthetic techniques. For example, a hydrophobic moiety can be attached to the N-terminus of the hydrophilic peptide. That is, hydrophobic electrophilic compounds (e.g., alkyl halide, carboxylic compound) can be reacted with the amine function.

In further examples, a hydrophobic moiety can be attached at the C-terminus of the hydrophilic peptide. Hydrophobic nucleophilic compounds (e.g., alcohol, amine, thiol) can be reacted with the carboxylic function present at the C-terminus to provide a covalent linkage (e.g., ester, amide, thioesters). It is further contemplated that the carboxylic function present at the C-terminus can be derivatized or reduced prior to reaction. For example, the carboxylic function can be reduced to form an alcohol and subsequently reacted with one or more hydrophobic electrophilic compounds (e.g., alkyl halide, carboxylic compound) to provide a covalent linkage (e.g., ether, ester).

As readily understood by those of skill in the art, peptide sequences can comprise peptide residues having one or more pendant groups. The pendant groups, in various aspects, can comprise one or more nucleophilic moieties (e.g., amine, hydroxyl, thiol) or one or more electrophilic moieties (e.g., carboxylic function). Such moieties can be reacted in a manner analogous to that disclosed above for N-terminus and C-terminus of a disclosed hydrophilic peptide.

Disclosed are methods of making the peptide amphiphile PA-C16-K5-NO, wherein the DS comprises one or more amino acid substitutions, comprising obtaining a peptide amphiphile comprising a nitric oxide (NO) producing donor sequence, wherein the peptide amphiphile comprises an N-terminus, alkylating the N-terminus of the peptide amphiphile with a hydrophobic moiety, wherein the alkylation comprises amination with a valeric acid, and conjugating NO to the peptide amphiphile. In some aspects, the peptide amphiphile that is obtained is the peptide amphiphile PA-C16-K5, wherein the DS comprises one or more amino acid substitutions, wherein the pendant amine groups can react with Nitric Oxide to form a diazeniumdiolate-modified peptide amphiphile.

Disclosed are methods of making a liposome comprising producing a lipid thin film, wherein the lipid thin film comprises cholesterol and lipids, wherein the lipids are 1,2-distearoyl-snglycero-3-phosphocholine (DSPC), 1,2-dipalmitoyl-snglycero-3-phosphocholine (DPPC), and 1,2-Distearoyl-sn-glycero-3-phosphoethanolamine-poly (ethylene glycol) (DSPE-PEG), or Dioleoyl-3-trimethylammonium propane (DOTAP); hydrating the lipid thin film with a buffer solution, wherein the buffer solution comprises a peptide amphiphile; and extruding the buffer solution using a polycarbonate membrane filters. In some aspects, the peptide amphiphile is the peptide amphiphile of any of those described herein, particularly PA-C16-K5-NO or PA-C16-K5, with one or more amino acid substitutions in the DS. In some aspects, extruding the buffer solution using a polycarbonate membrane filters comprises using 800 nm, 400 nm, and 200 nm polycarbonate membrane filters.

### I. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the peptide amphiphiles, compositions, liposomes, gels, medical devices, or combinations thereof described herein.

The disclosed kits can also include directions for making the medical devices coated in the disclosed peptide amphiphiles, compositions, gels or liposomes.

### Examples

### A. Example 1 Synthesis of PA-C16-GTAGLKKK-YIGSR (SEQ ID NO:19) and PA-C16-GTAKKKKK-YIGSR (SEQ ID NO:20)

PA-C16-GTAGLKKK-YIGSR (SEQ ID NO: 19) has been synthesized based on our established protocols. Specifically. PA-C16-GTAGLKKK-YIGSR (SEQ ID NO:19) consisting of the hydrophilic peptide sequence comprises an amino acid sequence Tyr-Ile-Gly-Ser-Arg (YIGSR; SEQ ID NO:2) and an amino acid sequence Gly-Thr-Ala-Gly-Leu-Lys-Lys-Lys (SEQ ID NO:4), which were synthesized using standard Fmoc-chemistry on an Advanced Chemtech Apex 396 peptide synthesizer, respectively. Alkylation was obtained by reacting N-termini of the peptides with 2 equivalents of palmitic acid. 2 equivalents of o-benzotriazole-N,N,N′,N′ tetramethyluroniumhexafluorophosphate (HBTU), and 4 equivalents of diisopropylethylamine (DiEA) in dimethylformamide (DMF) for 6 h at room temperature. Similarly. PA-C16-GTAKKKKK-YIGSR (SEQ ID NO:20), were synthesized using the abovementioned approaches. After repeating the alkylation reaction, cleavage and deprotection of peptide amphiphiles (PA) were performed using a mixture of trifluoroacetic acid (TFA), deionized (DI) water, triisopropylsilane, and anisole in the ratio of 90:1:1:1 for 3 hours at room temperature. The solution was concentrated using a rotary evaporator. PAs were precipitated in cold ether, collected, and dried under a vacuum. The crude PAs were dissolved in DI water at 1 wt % to make a PA solution.

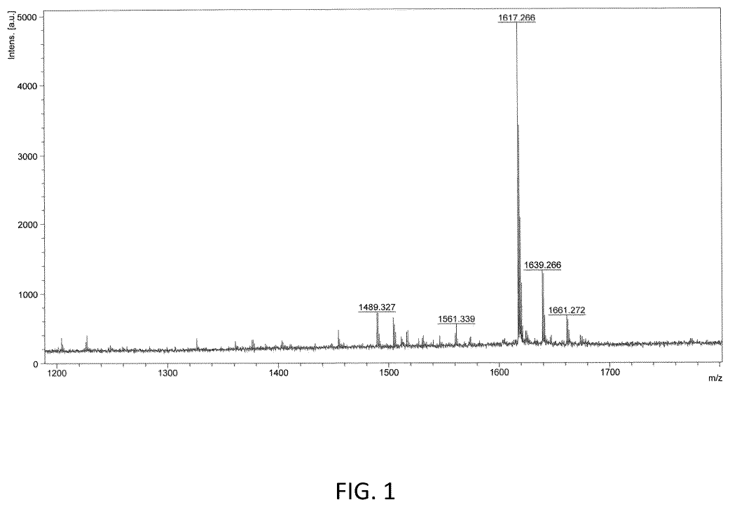

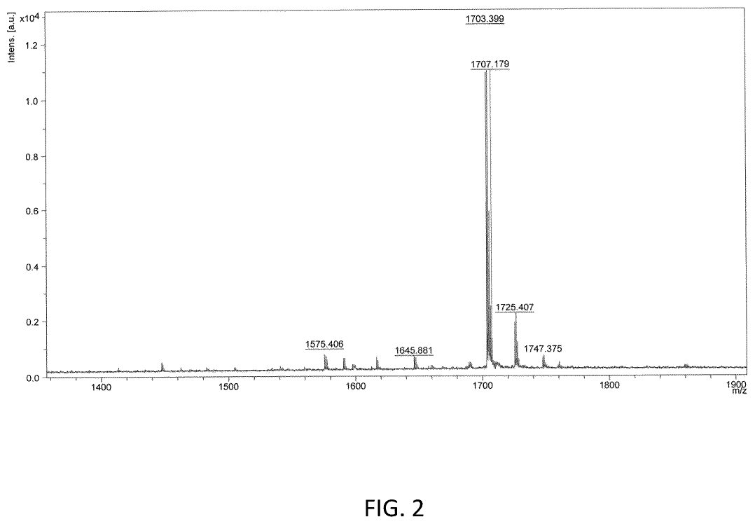

The PAs were analyzed by matrix-assisted laser desorption ionization time-of-flight (MALDI-TOF) mass spectrometry. Based on the MALDI-TOF result, the molecular weight of peptide amphiphile 11 (PA-C16-GTAGLKKK-YIGSR (SEQ ID NO: 19)) and peptide amphiphile 13 (PA-C16-GTAKKKKK-YIGSR (SEQ ID NO:20)), are approximately 1617.21 g/mol and 1703.4 g/mol, respectively (FIG. 1-FIG. 2).

In addition. 1 wt. % PA-C16-GTAGLKKK-YIGSR (SEQ ID NO:19) and PA-C16-GTAKKKKK-YIGSR (SEQ ID NO:20) solutions were diluted in DI water and cast onto carbon support mesh grids. The mesh grids were dried and negatively stained with 2% uranyl acetate. The prepared sample grids were then imaged on a FEI Tecnai T12 TEM microscope at 21000× and 52000× direct magnification.

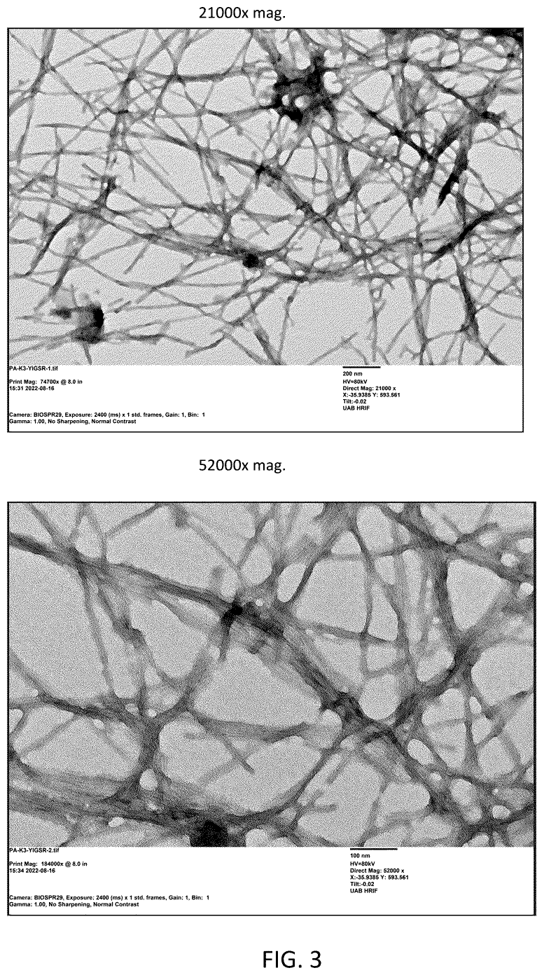

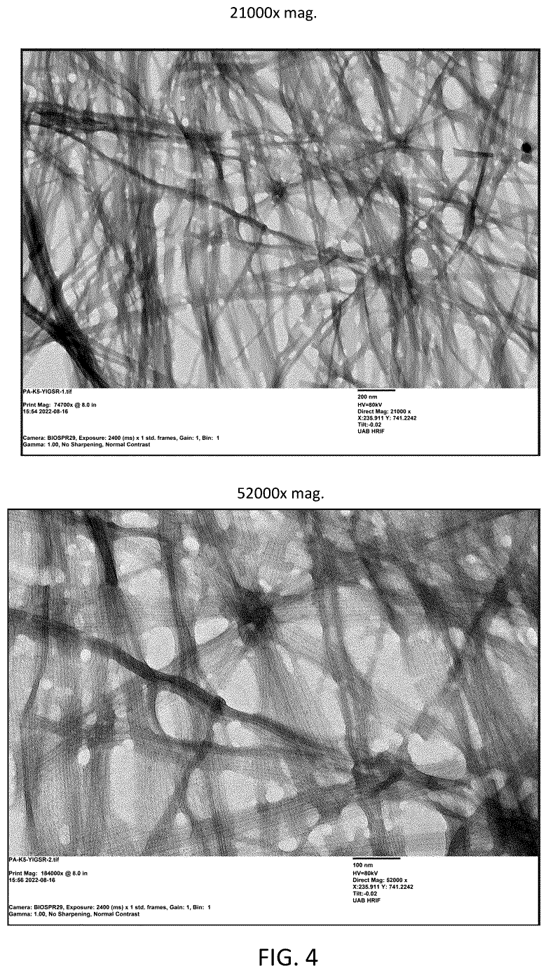

The TEM images of PA-C16-GTAGLKKK-YIGSR (SEQ ID NO: 19) and PA-C16-GTAKKKKK-YIGSR (SEQ ID NO:20) were taken, which are shown in FIG. 3-FIG. 4.

### B Example 2 Solubility and size of PA-C16-GTAGLKKK-YIGSR (SEQ ID NO: 19) and

(SEQ ID NO: 20)
PA-C16-GTAKKKKK-YIGSR

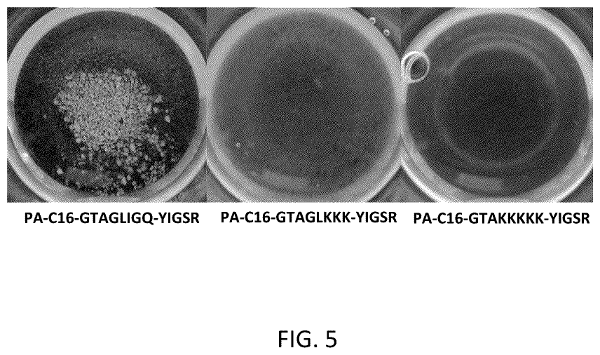

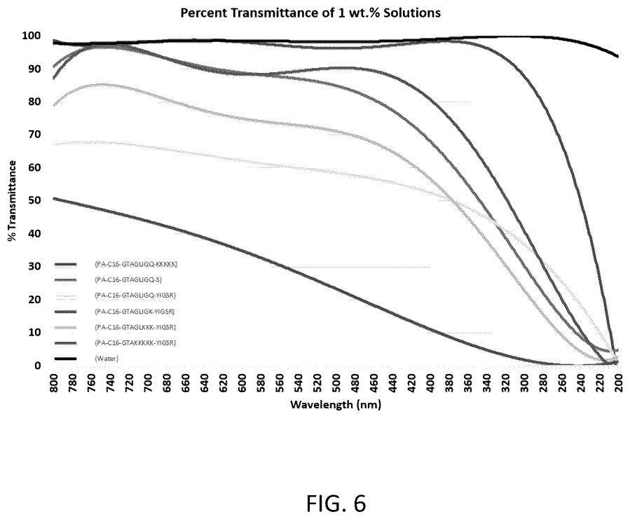

PA-C16-GTAGLKKK-YIGSR (SEQ ID NO: 19) and PA-C16-GTAKKKKK-YIGSR (SEQ ID NO:20) were dissolved in DI water at 1 wt %. Then, the solubility of PA-C16-GTAGLKKK-YIGSR (SEQ ID NO: 19) and PA-C16-GTAKKKKK-YIGSR (SEQ ID NO:20) were compared with PA-C16-GTAGLIGQ-YIGSR (SEQ ID NO:21 as seen in FIG. 5. As observed, PA-C16-GTAGLIGQ-YIGSR (SEQ ID NO:21) is insoluble and forms precipitates while PA-C16-GTAGLKKK-YIGSR (SEQ ID NO: 19) and PA-C16-GTAKKKKK-YIGSR (SEQ ID NO:20) form homogenous, transparent solutions. In addition, according to UV measurements, the percent transmittance of 1 wt % PA-C16-GTAGLKKK-YIGSR (SEQ ID NO: 19) and PA-C16-GTAKKKKK-YIGSR (SEQ ID NO:20) are 65% and 85%, as demonstrated by FIG. 6, which are significantly higher than unmodified PA-C16-GTAGLIGQ-YIGSR (˜45%). Higher percent transmittance indicates less opaqueness of solution, which corresponds with increased solubility of the peptide amphiphiles in DI water. Thus, PA-C16-GTAGLKKK-YIGSR (SEQ ID NO: 19) and PA-C16-GTAKKKKK-YIGSR (SEQ ID NO:20) are more soluble than unmodified PA-C16-GTAGLIGQ-YIGSR (SEQ ID NO:21).

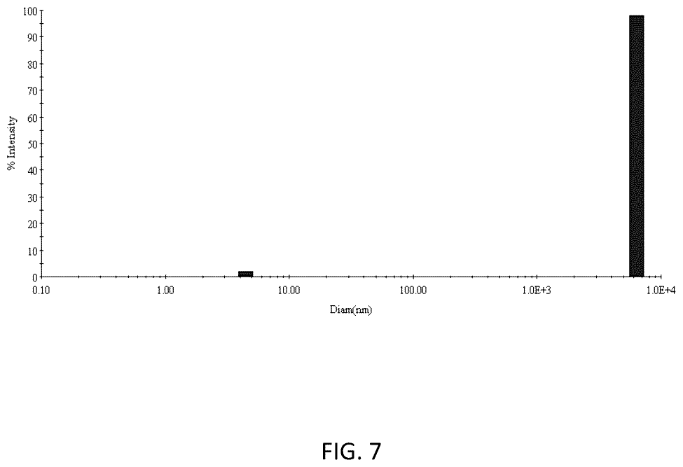

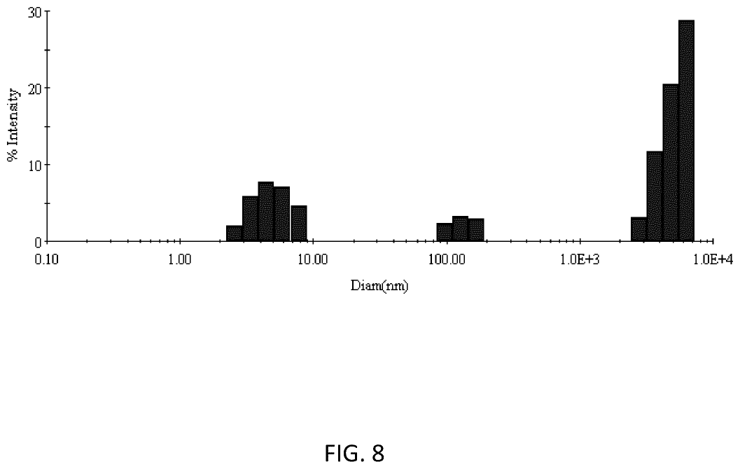

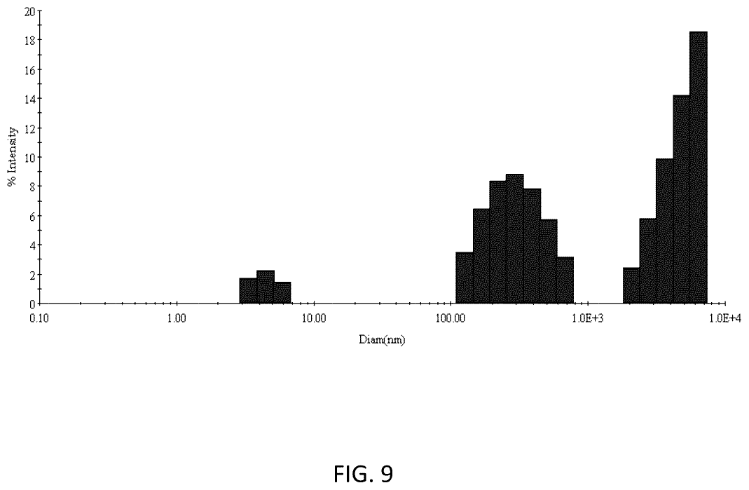

Additionally, 1 wt. % PA solutions were diluted equally and transferred into cuvettes. Then, the prepared cuvettes were analyzed by a ZetaSizer Nano ZS dynamic light scattering machine to estimate the particle sizes of the peptide amphiphiles in solution. According to the DLS data seen in FIG. 7-9, it is found that the radius of unmodified PA-C16-GTAGLIGQ-YIGSR (SEQ ID NO:21) is above 1 um, indicating large particle formation, while the radii of PA-C16-GTAGLKKK-YIGSR (SEQ ID NO: 19) and PA-C16-GTAKKKKK-YIGSR (SEQ ID NO:20) are 50±81 nm and 233±58.9 nm, respectively. These data shown here strongly indicate that PA-C16-GTAGLKKK-YIGSR (SEQ ID NO:19) and PA-C16-GTAKKKKK-YIGSR (SEQ ID NO:20) have significantly improved solubility than unmodified PA-C16-GTAGLIGQ-YIGSR (SEQ ID NO:21).

Those skilled in the art will recognize, or be able to ascertain using no more than routine experimentation, many equivalents to the specific embodiments of the method and compositions described herein. Such equivalents are intended to be encompassed by the following claims.

1.-2. (canceled)

3. A peptide amphiphile comprising a hydrophilic peptide sequence and a hydrophobic tail, wherein the hydrophobic tail comprises a moiety having a substituted C16 alkyl chain, wherein the hydrophilic peptide sequence comprises a degrading sequence (DS) wherein the degrading sequence (DS) comprises an amino acid sequence comprising the sequence (X₁X₂X₃X₄X₅X₆X₇X₈),
wherein X₁ is Gly, a positive amino acid, a negatively charged amino acid or a polar uncharged amino acid;
wherein X₂ is Thr, a positive amino acid, a negatively charged amino acid or a polar uncharged amino acid;
wherein X₃ is Ala, a positive amino acid, a negatively charged amino acid or a polar uncharged amino acid;
wherein X₄ is r Gly, a positive amino acid, a negatively charged amino acid or a polar uncharged amino acid;
wherein X₅ is Leu, a positive amino acid, a negatively charged amino acid or a polar uncharged amino acid;
wherein X₆ is Ile, a positive amino acid, a negatively charged amino acid or a polar uncharged amino acid;
wherein X₇ is Gly, a positive amino acid, a negatively charged amino acid or a polar uncharged amino acid; and
wherein X₈ is Gln. a positive amino acid, a negatively charged amino acid or a polar uncharged amino acid.

4. The peptide amphiphile of claim 3, wherein X₁ is Lys or Gly, X₂ is Lys or Thr, X₃ is Lys or Ala, X₄ is Lys or Gly, X₅ is Lys or Leu, X₆ is Lys or Ile, X₇ is Lys or Gly, and X₈ is Lys or Gln.

5. The peptide amphiphile of claim 3, wherein the degrading sequence (DS) comprises a MMP specific cleavage site.

6. The peptide amphiphile of claim 3, further comprising a cell-adhesive sequence.

7. The peptide amphiphile of claim 6, wherein the cell-adhesive sequence is an endothelial cell adhesive sequence that does not bind to smooth muscle cells and/or platelets.

8. The peptide amphiphile of claim 6, wherein the cell-adhesive sequence comprises the amino acid sequence YIGSR (SEQ ID NO:2).

9. The peptide amphiphile of any one of claims 1-8, wherein the DS comprises the amino acid sequence GTAGLIGK, GTAGLIKK, GTAGLKKK, GTAGKKKK, GTAKKKKK, GTKKKKKK, GKKKKKKK, or KKKKKKKK.

10.-16. (canceled)

17. The peptide amphiphile of claim 9, wherein one or more of the Lys residues comprise a pendant amine group.

18. The peptide amphiphile of claim 17, wherein the pendant amine group can react with Nitric Oxide to form a diazeniumdiolate-modified peptide amphiphile.

19. The peptide amphiphile claim 18, further comprising one or more molecules of Nitric Oxide.

20.-36. (canceled)

37. A liposome comprising the peptide amphiphile of claim 3.

38.-49. (canceled)

50. A gel comprising the peptide amphiphile of of claim 3.

51. A medical device coated with a peptide amphiphile claim 3.

52.-54. (canceled)

55. A method of treating arteriovenous fistula failure or cardiovascular disease in a subject, comprising administering to the subject a therapeutically effective amount of a composition comprising the peptide Amphiphile of claim 62 to a subject in need thereof.

56.-58. (canceled)

59. A method of preventing or treating cardiovascular disease in a subject, comprising administering to the subject a therapeutically effective amount of a composition comprising the peptide amphiphile of claim 62 to a subject in need thereof.

60. (canceled)

61. The peptide of claim 3, further comprising a nitric oxide producing donor sequence comprising the sequence of KKKKK (K5), and wherein one or more of the lysine residues comprise a pendant amine group

62. A peptide amphiphile comprising a hydrophilic peptide sequence and a hydrophobic tail,
wherein the hydrophobic tail comprises a moiety having a substituted C16 alkyl chain,
wherein the hydrophilic peptide sequence comprises:
a nitric oxide producing donor sequence comprising the sequence of KKKKK (K5), wherein one or more of the lysine residues of K5 comprise a pendant amine group, wherein one or more molecules of nitric oxide is bound to at least one pendant amine group, and
a degrading sequence (DS), wherein the DS comprises an amino acid sequence comprising the sequence (X₁X₂X₃X₄X₅X₆X₇X₈),
wherein X₁ is Gly, a positive amino acid, a negatively charged amino acid or a polar uncharged amino acid;
wherein X₂ is Thr, a positive amino acid, a negatively charged amino acid or a polar uncharged amino acid;
wherein X₃ is Ala, a positive amino acid, a negatively charged amino acid or a polar uncharged amino acid;
wherein X₄ is r Gly, a positive amino acid, a negatively charged amino acid or a polar uncharged amino acid;
wherein X₅ is Leu, a positive amino acid, a negatively charged amino acid or a polar uncharged amino acid;
wherein X₆ is Ile, a positive amino acid, a negatively charged amino acid or a polar uncharged amino acid;
wherein X₇ is Gly, a positive amino acid, a negatively charged amino acid or a polar uncharged amino acid; and
wherein X₈ is Gln, a positive amino acid, a negatively charged amino acid or a polar uncharged amino acid.

63. The peptide amphiphile of claim 62, further comprising a cell-adhesive sequence.

64. The peptide amphiphile of claim 63, wherein the cell-adhesive sequence comprises the amino acid sequence YIGSR (SEQ ID NO:2).

65. A composition comprising the peptide amphiphile of claim 62.
